(19) United States
(12) Patent Application Publication
(10) Pub. No.: US 2025/0000271 A1
(43) Pub. Date: Jan. 2, 2025

(54) Devices and Systems to Control Bed Temperature and Moisture Level

(71) Applicant: Robert A. Connor, St. Paul, MN (US)

(72) Inventor: Robert A. Connor, St. Paul, MN (US)

(21) Appl. No.: 18/827,987

(22) Filed: Sep. 9, 2024

Related U.S. Application Data
(63) Continuation-in-part of application No. 18/527,168, filed on Dec. 1, 2023.
Application No. 18/527,168 is a continuation-in-part of application No. 17/571,478, filed on Jan. 8, 2022, now Pat. No. 11,925,271.
Application No. 17/571,478 is a continuation-in-part of application No. 16/208,665, filed on Dec. 4, 2018, now abandoned.
Application No. 16/208,665 is a continuation-in-part of application No. 14/703,916, filed on May 5, 2015, now Pat. No. 10,179,064.
(60) Provisional application No. 63/604,816, filed on Nov. 30, 2023.
(60) Provisional application No. 63/441,818, filed on Jan. 29, 2023.
(60) Provisional application No. 61/991,172, filed on May 9, 2014.

(51) Int. Cl. A47C 21/04; A47C 27/08; A47G 9/02; F24F 5/00

(52) U.S. Cl. CPC A47C 21/044; A47C 27/085; A47G 9/0215; F24F 5/0096

(57) ABSTRACT

Disclosed herein are devices and systems to control and customize the temperature and moisture levels of a bed. A device or system can include: intra-room channels which conduct a flowable substance between a central HVAC vent and a bed; and intra-bed channels which conduct a flowable substance through a bedding component such as a bed mattress, bed mattress pad or topper, lower/fitted bed sheet, upper bed sheet, blanket, comforter, or quilt. A device or system can be a hybrid system wherein a liquid flow channel is nested inside an air flow channel.

### CROSS-REFERENCE TO RELATED APPLICATIONS

This application is a continuation-in-part of U.S. patent application Ser. No. 18/527,168 filed on 2023 Dec. 1.

U.S. patent application Ser. No. 18/527,168 claimed the priority benefit of U.S. Provisional Patent Application 63/604,816 filed on 2023 Nov. 30. U.S. patent application Ser. No. 18/527,168 claimed the priority benefit of U.S. Provisional Patent Application 63/441,818 filed on 2023 Jan. 29. U.S. patent application Ser. No. 18/527,168 was a continuation-in-part of U.S. patent application Ser. No. 17/571,478 filed on 2022 Jan. 8.

U.S. patent application Ser. No. 17/571,478 was a continuation-in-part of U.S. patent application Ser. No. 16/208,665 filed on 2018 Dec. 4. U.S. patent application Ser. No. 16/208,665 was a continuation-in-part of U.S. patent application Ser. No. 14/703,916 filed on 2015 May 5 which issued as U.S. Pat. No. 10,179,064 on 2019 Jan. 15. U.S. patent application Ser. No. 14/703,916 claimed the priority benefit of U.S. Provisional Patent Application 61/991,172 filed on 2014 May 9.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to devices and systems for controlling bed temperature and moisture level.

### INTRODUCTION

Devices and systems which enable people to manage their sleep environments (including customization and control of bed temperature and moisture/humidity level) can improve their sleep quality and overall health. There has been considerable innovation during the past decade with respect to devices and systems for modification and management of sleep environments. However, some challenges remain. For example, cooling can be more challenging than heating. Extracted heat has to go somewhere. Also, devices to control bed temperature and humidity using air flow and devices to control bed temperature and humidity using liquid flow each have their pros and cons. For example, liquid flow can be better for thermal transfer and air flow can be better for removing moisture. To address these challenges, disclosed herein are devices and systems which can more effectively manage sleep environments, including control of bed temperature and moisture/humidity level.

### REVIEW OF THE RELEVANT ART

U.S. patent applications 20150352313 (Franceschetti et al., Dec. 10, 2015, “Methods and Systems for Gathering Human Biological Signals and Controlling a Bed Device”), 20150355605 (Franceschetti et al., Dec. 10, 2015, “Methods and Systems for Gathering and Analyzing Human Biological Signals”), 20170259028 (Franceschetti et al., Sep. 14, 2017, “Methods and Systems for Gathering and Analyzing Human Biological Signals”), 20190269878 (Franceschetti et al., Sep. 5, 2019, “Methods and Systems for Gathering and Analyzing Human Biological Signals”), and 20190321581 (Franceschetti et al., Oct. 24, 2019, “Methods and Systems for Gathering and Analyzing Human Biological Signals”), as well as U.S. Pat. No. 9,186,479 (Franceschetti et al., Nov. 17, 2015, “Methods and Systems for Gathering Human Biological Signals and Controlling a Bed Device”), U.S. Pat. No. 9,981,107 (Franceschetti et al., May 29, 2018, “Methods and Systems for Gathering and Analyzing Human Biological Signals”), and U.S. Pat. No. 10,792,461 (Franceschetti et al., Oct. 6, 2020, “Methods and Systems for Gathering and Analyzing Human Biological Signals”) disclose using sensors to measure biometric parameters such as body temperature and heart rate and using this information to adjust the temperature of a bed and/or determine when to wake a person.

U.S. patent applications 20160073950 (Franceschetti et al., Mar. 17, 2016, “Vibrating Alarm System and Operating Methods”), 20160136383 (Franceschetti et al., May 19, 2016, “Vibrating Pillow Strip and Operating Methods”), and 20170296773 (Franceschetti et al., Oct. 19, 2017, “Vibrating Pillow Strip and Operating Methods”), as well as U.S. Pat. No. 9,586,021 (Franceschetti et al., Mar. 7, 2017, “Vibrating Pillow Strip and Operating Methods”) disclose methods and systems for: gathering human biological signals, such as heart rate, respiration rate, or temperature; analyzing the gathered human biological signals; and controlling a vibrating pillow strip based on the analysis.

U.S. patent applications 20160128488 (Franceschetti et al., May 12, 2016, “Apparatus and Methods for Heating or Cooling a Bed Based on Human Biological Signals”), 20160310697 (Franceschetti et al., Oct. 27, 2016, “Bed Device System and Methods”), 20170028165 (Franceschetti et al., Feb. 2, 2017, “Bed Device System and Methods”), and 20200390998 (Franceschetti et al., Dec. 17, 2020, “Apparatus and Methods for Heating or Cooling a Bed Based on Human Biological Signals”), as well as U.S. Pat. No. 9,694,156 (Franceschetti et al., Jul. 4, 2017, “Bed Device System and Methods”) disclose methods and systems for an adjustable bed device which is heated or cooled based on biological signals from multiple users. U.S. patent applications 20170135632 (Franceschetti et al., May 18, 2017, “Detecting Sleeping Disorders”) and 20190254593 (Franceschetti et al., Aug. 22, 2019, “Detecting Sleeping Disorders”), as well as U.S. Pat. No. 10,105,092 (Franceschetti et al., Oct. 23, 2018, “Detecting Sleeping Disorders”) disclose automatic adjustment of a bed in response to snoring or sleep apnea.

U.S. patent applications 20170135881 (Franceschetti et al., May 18, 2017, “Adjustable Bedframe and Operating Methods”), 20170135882 (Franceschetti et al., May 18, 2017, “Adjustable Bedframe and Operating Methods for Health Monitoring”), and 20170135883 (Franceschetti et al., May 18, 2017, “Adjustable Bedframe and Operating Methods”), as well as U.S. Pat. No. 10,154,932 (Franceschetti et al., Dec. 18, 2018, “Adjustable Bedframe and Operating Methods for Health Monitoring”) disclose adjustment of a bed frame based on biological signals from multiple users. U.S. patent application No. 20160073788 (Franceschetti et al., Mar. 17, 2016, “Sensor Strip for Gathering Human Biological Signals and Controlling a Bed Device”) discloses methods and systems for an adjustable bed device which is heated or cooled based on biological signals from multiple users.

U.S. patent application No. 20200405998 (Franceschetti et al., Dec. 31, 2020, “Sleep Pod”) discloses sleep pods which provide a personalized sleeping experience based on an analysis of biological signals, environmental characteristics, occupant history, and other factors. U.S. patent application No. 20200397379 (Franceschetti et al., Dec. 24, 2020, “Systems and Methods for Detecting a Biological Signal of a User of an Article of Furniture”) discloses a flexible sensor device on furniture which detects one or more biological signals of a user of the furniture. U.S. patent application No. 20210315389 (Franceschetti et al., Oct. 14, 2021, “Systems and Methods for Regulating a Temperature of an Article of Furniture”) discloses a system that adjusts a mattress position in response to a biological signal associated with a user.

U.S. patent applications 20100011502 (Brykalski et al., Jan. 21, 2010, “Climate Controlled Bed Assembly”), 20120227182 (Brykalski et al., Sep. 13, 2012, “Climate Controlled Bed Assembly”), and 20130227783 (Brykalski et al., Sep. 5, 2013, “Environmentally Conditioned Bed Assembly”), as well as U.S. Pat. No. 8,418,286 (Brykalski et al., Apr. 16, 2013, “Climate Controlled Bed Assembly”) and U.S. Pat. No. 8,181,290 (Brykalski et al., May 22, 2012, “Climate Controlled Bed Assembly”) disclose a climate controlled bed includes an upper portion comprising a core with a top core surface and a bottom core surface. U.S. Pat. No. 9,125,497 (Brykalski et al., Sep. 8, 2015, “Climate Controlled Bed Assembly with Intermediate Layer”) and U.S. Pat. No. 9,974,394 (Brykalski et al., May 22, 2018, “Climate Controlled Bed Assembly with Intermediate Layer”) disclose a climate controlled bed with an upper portion or mattress having at least one fluid distribution member in fluid communication with an internal passageway.

U.S. Pat. No. 8,332,975 (Brykalski et al., Dec. 18, 2012, “Climate-Controlled Topper Member for Medical Beds”) discloses a conditioner mat with an upper layer having openings, a lower layer being fluid impermeable, an interior chamber defined by the upper layer and the lower layer, and a spacer material within the interior chamber. U.S. patent application No. 20130097776 (Brykalski et al., Apr. 25, 2013, “Thermally Conditioned Bed Assembly”) and U.S. Pat. No. 9,603,459 (Brykalski et al., Mar. 28, 2017. “Thermally Conditioned Bed Assembly”) disclose a bed with a flow conditioning member in a recessed area of a cushion and a fluid temperature regulation system.

U.S. patent applications 20180000255 (Youngblood et al., Jan. 4, 2018, “Article Comprising a Temperature-Conditioned Surface, Thermoelectric Control Unit, and Method for Temperature-Conditioning the Surface of an Article”), 20200046134 (Youngblood et al., Feb. 13, 2020, “Article Comprising a Temperature-Conditioned Surface, Thermoelectric Control Unit, and Method for Temperature-Conditioning the Surface of an Article”), and 20210267379 (Youngblood et al., Sep. 2, 2021. “Article Comprising a Temperature-Conditioned Surface, Thermoelectric Control Unit, and Method for Temperature-Conditioning the Surface of an Article”) as well as U.S. Pat. No. 10,986,933 (Youngblood et al., Apr. 27, 2021, “Article Comprising a Temperature-Conditioned Surface, Thermoelectric Control Unit, and Method for Temperature-Conditioning the Surface of an Article”) and U.S. Pat. No. 1,013,338 (Youngblood et al., May 25, 2021, “Article Comprising a Temperature-Conditioned Surface, Thermoelectric Control Unit, and Method for Temperature-Conditioning the Surface of an Article”) disclose systems, methods, and articles for temperature conditioning a surface.

U.S. patent applications 20180110960 (Youngblood et al., Apr. 26, 2018, “Stress Reduction and Sleep Promotion System”), 20200077942 (Youngblood et al., Mar. 12, 2020, “Stress Reduction and Sleep Promotion System”), 20210219736 (Youngblood et al., Jul. 22, 2021, “System for Heat Exchange with a Circulating Fluid”), 20210219737 (Youngblood et al., Jul. 22, 2021, “Article and System for Heating or Cooling a Surface”), and 20210268226 (Youngblood et al., Sep. 2, 2021, “Stress Reduction and Sleep Promotion System”), as well as U.S. Pat. No. 11,013,883 (Youngblood et al., May 23, 2021. “Stress Reduction and Sleep Promotion System”) disclose systems, methods, and articles for stress reduction and sleep promotion, including modification of bed temperature.

U.S. patent application No. 20210235880 (Youngblood et al., Aug. 5, 2021, “Article Comprising a Temperature-Conditioned Surface, Thermoelectric Control Unit, and Method for Temperature-Conditioning the Surface of an Article”) and U.S. Pat. No. 10,278,511 (Youngblood et al., May 7, 2019, “Article Comprising a Temperature-Conditioned Surface, Thermoelectric Control Unit, and Method for Temperature-Conditioning the Surface of an Article”) and U.S. Pat. No. 11,013,339 (Youngblood et al., May 25, 2021, “Article Comprising a Temperature-Conditioned Surface, Thermoelectric Control Unit, and Method for Temperature-Conditioning the Surface of an Article”) disclose a thermoelectric control unit for regulating liquid temperature in a hydraulic circuit.

U.S. Pat. No. 10,986,934 (Youngblood et al., Apr. 27, 2021, “Multi-Zone Temperature Modulation System for Bed or Blanket”) and U.S. Pat. No. 11,147,389 (Youngblood et al., Oct. 19, 2021, “Multi-Zone Temperature Modulation System for Bed or Blanket”) disclose a temperature modulation system for a bed, blanket, or other furniture which includes a fluid for moderating temperature change, a number of conduit circuits for directing the fluid through respective zones, a control unit including a thermoelectric device for modulating temperature of the fluid, and a pump. U.S. patent application No. 20210100378 (Youngblood et al., Apr. 8, 2021, “Weighted Blanket with Thermally Regulated Fluid”) discloses a weighted article for assisting sleep, wherein the weighted article includes heating and cooling means.

U.S. Pat. No. 6,006,524 (Park, Dec. 28, 1999, “Temperature Controller for Bedding”) discloses a temperature controller for bedding which can always provide a comfortable sleeping environment by maintaining bedding at a temperature suitable for the human body during sleeping by supplying cold or warm heat transfer medium to the inside of bedding. U.S. patent application No. 20140276227 (Perez, Sep. 18, 2014, “Sleep Management Implementing a Wearable Data-Capable Device for Snoring-Related Conditions and Other Sleep Disturbances”) discloses a wearable device which vibrates to reduce snoring. U.S. patent application No. 20110115635 (Petrovski et al., May 19, 2011, “Control Schemes and Features for Climate-Controlled Beds”) and U.S. Pat. No. 8,893,329 (Petrovski et al., Nov. 24, 2014, “Control Schemes and Features for Climate-Controlled Beds”) disclose a climate-conditioned bed with an upper portion having at least a first climate zone and at least one fluid module associated with such a first climate zone.

U.S. patent application No. 20070193278 (Polacek et al., Aug. 23, 2007, “Cooling Device and Method”) discloses a device for providing cooling to a body in response to a measured change in temperature of the body's skin surface. U.S. patent application No. 20140207292 (Ramagem et al., Jul. 24, 2014, “Method and System to Control Thermostat Using Biofeedback”) discloses methods and systems for receiving physiological data of occupants of a building and using the information to control or regulate a controllable setpoint of a climate-control system for the building. U.S. patent application No. 20140201910 (Rand, Jul. 24, 2014, “Tunnel Generating Bed Cooling System”) discloses a skin surface cooling system used in bed during sleep which directs forced room air to an area above a flat mattress and under a top bed sheet, creating a tunnel of cool, moving air. U.S. patent application No. 20120138067 (Rawls-Meehan, Jun. 7, 2012, “System and Method for Mitigating Snoring in an Adjustable Bed”) discloses a bed which moves to shift a person to an anti-snoring position when the person snores.

U.S. patent application No. 20090287063 (Freedman et al., Nov. 19, 2009, “Hygrometric Determination of Hot Flashes”) discloses a method of measuring hot flashes based on monitoring skin moisture. U.S. Pat. No. 7,640,764 (Gammons et al., Jan. 5, 2010, “Portable Coolant System”) and U.S. patent application No. 20080060374 (Gammons et al., Mar. 13, 2008, “Portable Coolant System”) disclose a portable cooling system with ice. U.S. patent application No. 20100204764 (Garetz, Aug. 12, 2010, “Method for Treating Hot Flashes Associated with Menopause During Sleep”) discloses a method for detecting a hot flash using a skin temperature or humidity sensor and circulating cooling fluid through a vest. U.S. Pat. No. 8,755,879 (Hang et al., Jun. 17, 2014, “Sleep Tracking and Waking Optimization System and Method Therefor”) discloses EEG and pressure sensors which need not be attached to a person's head. U.S. Pat. No. 9,888,782 (Jannke, Feb. 13, 2018, “Temperature Controlled Mattress System”) discloses a mattress assembly with at least one channel which receives temperature conditioned air flow.

U.S. patent application No. 20110295083 (Doelling et al., Dec. 1, 2011, “Devices, Systems, and Methods for Monitoring, Analyzing, and/or Adjusting Sleep Conditions”) discloses therapeutic and diagnostic systems and methods to help an individual with a sleep disordered breathing condition. U.S. patent application No. 20120152260 (Flinsenberg et al., Jun. 21, 2012, “Snoring Reduction Apparatus”) discloses an apparatus for shifting a person to a different sleep position when they snore.

U.S. patent application No. 20130036549 (McKlarney, Feb. 14, 2013, “System for Cooling a Body Useful for Reducing the Effect of Hot Flashes”) and U.S. Pat. No. 8,887,328 (McKlarney, Nov. 18, 2014, “System for Cooling a Body Useful for Reducing the Effect of Hot Flashes”) disclose systems, methods, and devices for automatically detecting an imminent a hot flash and addressing related symptoms via cooling. U.S. Pat. No. 10,051,973 (Morgan et al., Aug. 21, 2018, “Air Conditioned Mattresses”) discloses an air conditioned mattress with at least one encasement configured to receive a conditioned flow of air. U.S. patent application No. 20080155750 (Mossbeck, Jul. 3, 2008, “Anti-Snore Bedding Having Adjustable Portions”) discloses a bed whose configuration changes when a person snores. U.S. patent application No. 20140047644 (Mossbeck, Feb. 20, 2014, “Anti-Snore Bed Having Inflatable Members”) discloses bedding with inflatable members which responds to snoring.

U.S. patent application No. 20140222174 (Teller et al., Aug. 7, 2014, “Wearable Apparatus to Detect and Monitor Sleep and Other Activities”) discloses methods and apparatuses for measuring a state parameter of an individual using signals based on one or more sensors. U.S. patent application 20140323799 (Van Driel et al., Oct. 30, 2014, “System and a Method for Improving a Person's Sleep”) discloses a system and a method for improving a person's sleep with a bedding layer comprising a plurality of individually controllable thermally adjustable zones and a plurality of temperature sensors. U.S. Pat. No. 9,877,593 (Van Erlach, Jan. 30, 2018, “Smart Surface for Sleep Optimization”), application No. 20160045035 (Van Erlach, Feb. 18, 2016, “Smart Surface for Sleep Optimization”), and application No. 20180132627 (Van Erlach, May 17, 2018, “Smart Surface for Sleep Optimization”) disclose the delivery of therapy to a body based on information from two sets of sensors in contact with the body. U.S. patent applications 20130131464 (Westbrook et al., May 23, 2013, “System for the Assessment of Sleep Quality in Adults and Children”) and 20100240982 (Westbrook et al., Sep. 23, 2010, “System for the Assessment of Sleep Quality in Adults and Children”) disclose systems and methods for assessment of sleep quality which include a sensor strip and a nasal mask to obtain physiological signals.

U.S. patent application No. 20160015315 (Auphan et al., Jan. 21, 2016, “System and Method to Monitor and Assist Individual's Sleep”) discloses a sleep assist system with a loudspeaker, light source, temperature sensor, and control unit. U.S. patent application No. 20060162074 (Bader, Jul. 27, 2006, “Device and Method for Controlling Physical Properties of a Bed”) discloses a bed whose properties are adjusted based on the state of a person on the bed. U.S. Pat. No. 8,932,199 (Berka et al., Jan. 13, 2015, “Systems and Methods for Optimization of Sleep and Post-Sleep Performance”) discloses a sleep mask with electromagnetic sensors which awakens a sleeping person at an appropriate sleep stage. U.S. Pat. No. 8,628,462 (Berka et al., Jan. 14, 2014, “Systems and Methods for Optimization of Sleep and Post-Sleep Performance”) discloses systems for monitoring a person's sleep and generating sensory stimuli.

U.S. patent application No. 20160338871 (Nunn et al., Nov. 24, 2016, “Inflatable Air Mattress Snoring Detection and Response”) and U.S. Pat. No. 9,370,457 (Nunn et al., Jun. 21, 2016, “Inflatable Air Mattress Snoring Detection and Response”) disclose a bed whose firmness is changed in response to snoring. U.S. patent application No. 20170003666 (Nunn et al., Jan. 5, 2017, “Automation for Improved Sleep Quality”) discloses using historical sleep metrics and sensor data to create a corrective plan that specifies a change to an environmental control system. U.S. patent applications 20110289684 (Parish et al., Dec. 1, 2011, “System and Method for Thermoelectric Personal Comfort Controlled Bedding”), 20110314837 (Parish et al., Dec. 29, 2011, “System and Method for Thermoelectric Personal Comfort Controlled Bedding”), and 20120000207 (Parish et al., Jan. 5, 2012, “System and Method for Thermoelectric Personal Comfort Controlled Bedding”) disclose a system and method for controlling ventilation in a bed (mattress) including a user-controlled air conditioning control system.

U.S. patent applications 20160361515 (Jung et al., Dec. 15, 2016, “Method and Apparatus for Controlling Temperature Adjustment Device”) and 20190269883 (Jung et al., Sep. 5, 2019, “Method and Apparatus for Controlling Temperature Adjustment Device”) disclose a method and apparatus for controlling a temperature adjustment device using a sensing device. U.S. patent application 20130234823 (Kahn et al., Sep. 12, 2013, “Method and Apparatus to Provide an Improved Sleep Experience”) discloses using sound to guide person to a desired sleep state. U.S. patent application 20170027498 (Larson et al., Feb. 2, 2017, “Devices, Systems, and Methods for Preventing, Detecting, and Treating Pressure-Induced Ischemia, Pressure Ulcers, and Other Conditions”) discloses bed sensors for monitoring biometric parameters of a patient in bed and suggesting moving the patient when appropriate. U.S. Pat. No. 8,402,579 (Marquette et al., Mar. 26, 2013, “Climate Controlled Beds and Methods of Operating the Same”) discloses a climate controlled seat, bed or other assembly with a blower and two or more thermoelectric devices or other conditioning fluid modules.

U.S. Pat. No. 9,907,929 (Rink et al., Mar. 6, 2018, “Method and Device for Monitoring and Treating Sleep Disorders and Sleep-Related Conditions”) discloses methods and devices for monitoring a sleeping person and partially awakening the person during a sleep terror. U.S. Pat. No. 11,103,081 (Sherman et al., Aug. 31, 2021, “Climate Controlled Mattress System”) discloses a climate control system with a heating mechanism disposed between one or more foam layers of structure and a separate cooling mechanism disposed in the foam layers. U.S. Pat. No. 8,984,687 (Stusynski et al., Mar. 24, 2015, “Partner Snore Feature for Adjustable Bed Foundation”) and U.S. Pat. No. 10,058,467 (Stusynski et al., Aug. 28, 2018, “Partner Snore Feature for Adjustable Bed Foundation”), as well as U.S. patent applications 20140259419 (Stusynski et al., Sep. 18, 2014, “Partner Snore Feature for Adjustable Bed Foundation”) and 20150157519 (Stusynski et al., Jun. 11, 2015, “Partner Snore Feature for Adjustable Bed Foundation”) disclose a bed with two sections, wherein a person on a first section can control the articulation of a second section.

U.S. Pat. No. 9,955,791 (Chandler et al., May 1, 2018, “Climate Controlled Mattress Assembly and Related Method”) discloses mattress assemblies that use air bladders to provide climate control. U.S. patent application No. 20180000633 (Coleman et al., Jan. 4, 2018, “Microclimate Management System with Wireless Sensors”) discloses a method for monitoring a patient on a support device including: receiving a temperature reading from a wireless sensor coupled to the patient; comparing the temperature reading to air flowing through an airflow system associated with the patient support device; and modifying the air flowing through the airflow system. U.S. patent application 20100099954 (Dickinson et al., Apr. 22, 2010, “Data-Driven Sleep Coaching System”) discloses a system for monitoring a person's EEG to improve their sleep.

U.S. patent application No. 20180310719 (Wiggermann, Nov. 1, 2018, “Adaptable Mattress”) discloses a mattress with a phase changeable component and a thermal management system which controls the temperature of the phase changeable component to change the firmness of the mattress. U.S. Pat. No. 8,836,516 (Wolfe et al., Sep. 16, 2014, “Snoring Treatment”) discloses devices for detecting and reducing snoring including microphones and motion sensors. U.S. Pat. No. 5,555,579 (Wu, Sep. 17, 1996, “Mattress Assembly with Semiconductor Thermo-Control”) discloses a mattress with a network of grooves covered within a soft covering layer, a control box, and a water pipes within the network of grooves and connected to hot and cold water reservoirs inside the control box. U.S. patent application No. 20160007914 (Xu et al., Jan. 14, 2016, “Sleep Control Device”) discloses systems and methods for stimulating a person when they snore to cause them to shift their sleep position.

### SUMMARY OF THE INVENTION

Disclosed herein are devices and systems to control and customize the climate (e.g. the temperature and moisture/humidity level) of a bed. These devices and systems can include: one or more intra-room flow channels which conduct a flowable substance between a central HVAC (Heating Ventilation and Air Conditioning) vent and a bed; and one or more intra-bed flow channels which conduct a flowable substance through a bedding component such as a bed mattress, bed mattress pad or topper, lower/fitted bed sheet, upper bed sheet, blanket, comforter, or quilt. In an example, both flowable substances can be air. In another example, both flowable substances can be a liquid. In an example, the flowable substance in an intra-room flow channel can be different than the flowable substance in an intra-bed flow channel.

In an example, a device or system can be a hybrid device or system which uses both air flows and liquid flows. In an example, intra-bed flow channels can conduct liquid through an interior flow channel and can conduct air through an outer flow channel, wherein the interior flow channel is nested within the outer flow channel. Such a hybrid (air flow and liquid flow) system can combine the temperature-control efficiency of liquid flow and the moisture-removing benefit of air flow. Disclosed herein is also a leak detection and reduction mechanism for devices or systems with liquid flows.

### BRIEF INTRODUCTION TO THE FIGURES

FIGS. 1 and 2 show a device or system which controls bed climate using: an intra-room flow channel which spans along a wall between an HVAC vent and a bedding component (e.g. mattress, mattress pad or topper, lower and/or fitted bed sheet, upper bed sheet, blanket, comforter, or quilt); and intra-bed flow channels in the bedding component.

FIGS. 3 and 4 show a device or system which controls bed climate using: an intra-room flow channel which spans along a floor between an HVAC vent and a bedding component (e.g. mattress, mattress pad or topper, lower and/or fitted bed sheet, upper bed sheet, blanket, comforter, or quilt); and intra-bed flow channels in the bedding component.

FIGS. 5 and 6 show a device or system which controls bed climate using: an intra-room flow channel with modular sections between an HVAC vent and a bedding component (e.g. mattress, mattress pad or topper, lower and/or fitted bed sheet, upper bed sheet, blanket, comforter, or quilt); and intra-bed flow channels in the bedding component.

FIGS. 7 and 8 show a device or system which controls bed climate using: an intra-room flow channel between an HVAC vent near a bedroom ceiling and a bedding component (e.g. mattress, mattress pad or topper, lower and/or fitted bed sheet, upper bed sheet, blanket, comforter, or quilt); and intra-bed flow channels in the bedding component.

FIGS. 9 and 10 show a device or system which controls bed climate using: an intra-room flow channel between an HVAC vent on a bedroom floor and a bedding component (e.g. mattress, mattress pad or topper, lower and/or fitted bed sheet, upper bed sheet, blanket, comforter, or quilt); and intra-bed flow channels in the bedding component.

FIGS. 11 and 12 show views at two different times of a device or system which controls bed climate including a plurality of individually-adjustable flow valves around the perimeter of a bed.

FIG. 13 shows a device or system which controls bed climate including a flow-accelerating component near a bed.

FIG. 14 shows a device or system which controls bed climate including a flow-accelerating component near a central HVAC vent.

FIG. 15 shows a device or system which controls bed climate using liquid flow in intra-room channels between a bed and a central HVAC vent and air flow in intra-bed channels in a bedding component.

FIG. 16 shows a device or system which controls bed climate using liquid flow in intra-room channels between a bed and a central HVAC vent and also liquid flow in intra-bed channels in a bedding component.

FIG. 17 shows a hybrid device or system which controls bed climate with a liquid flow channel nested within an air flow channel.

FIG. 18 shows a cross-sectional view of a liquid flow channel nested within a lower portion of an air flow channel.

FIG. 19 shows a cross-sectional view of two liquid flow channels nested within an air flow channel.

FIGS. 20 and 21 shows views at two different times of a bedding component wherein air flow channels under a person's body release more air than channels which are not under the person's body.

FIGS. 22 and 23 shows a device or system which controls the bed climate including a plurality of valves.

FIG. 24 shows an intra-room flow channel whose cross-sectional shape is arcuate and convex.

FIG. 25 shows an intra-room flow channel whose cross-sectional shape is quadrilateral.

FIG. 26 shows a wall-based intra-room flow channel whose cross-sectional shape is rounded-quadrilateral.

FIG. 27 shows a floor-based intra-room flow channel whose cross-sectional shape is rounded-quadrilateral.

FIG. 28 shows an intra-room flow channel housing two arcuate flow sub-channels.

FIG. 29 shows an intra-room flow channel housing two quadrilateral flow sub-channels.

FIGS. 30 through 32 show views at three different times of a valve that adjusts how much air goes from a vent to an intra-room flow channel versus how much air goes from the vent into ambient bedroom air.

FIGS. 33 and 34 show a device or system which controls bed climate with intra-bed flow channels around a portion of a bed perimeter.

FIG. 35 shows a device or system which controls bed climate with intra-bed flow channels and valves around a portion of a bed perimeter.

FIGS. 36 through 38 show a leakage reduction mechanism comprising valves which close in response to leakage detected by a moisture sensor.

### DETAILED DESCRIPTION OF THE FIGURES

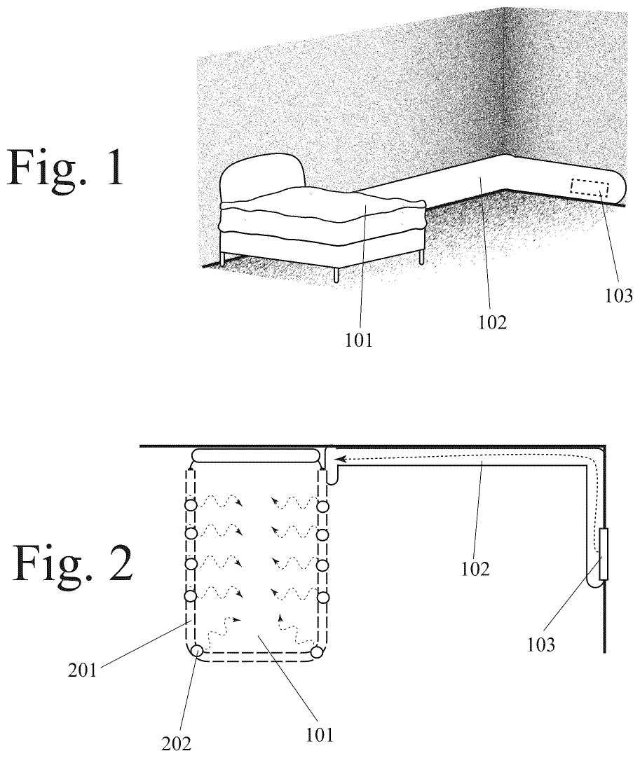

Before discussing the specific examples of devices for sleep environment control and management which are shown in FIGS. 1 through 38, it is useful to first discuss some introductory concepts and component variations. Having such an introductory section helps to avoid repeating these concepts and variations in the narratives accompanying each of the figures. However, it is to be understood that these concepts and component variations can be selectively applied to the specific examples shown in FIGS. 1 through 38 where relevant.

In an example, a device or system to control the climate of one or more areas of a bed can comprise: one or more intra-room flow channels which conduct a flowable substance between a first component and a second component in a bedroom; wherein the first component is selected from the group consisting of (a) an airflow vent though which air enters the bedroom from a central HVAC (Heating Ventilation and Air Conditioning) system, (b) an airflow vent through which air exits the bedroom to a central HVAC system, and (c) an air conditioner mounted in a bedroom window; and wherein the second component is a bedding component; and one or more intra-bed flow channels which conduct a flowable substance through the bedding component in order to change the climate of one or more areas of a bed.

In an example, the flowable substance conducted by one or more intra-room flow channels can be air and the flowable substance conducted by one or more intra-bed flow channels can also be air. In an example, the flowable substance conducted by one or more intra-room flow channels can be a liquid and the flowable substance conducted by one or more intra-bed flow channels can also be the liquid. In an example, the flowable substance conducted by one or more intra-room flow channels can be air and the flowable substance conducted by one or more intra-bed flow channels can be a liquid. In an example, the flowable substance conducted by one or more intra-room flow channels can be a liquid and the flowable substance conducted by one or more intra-bed flow channels can be air.

In an example, the device or system can further comprise a valve which changes the proportion of air from a central HVAC vent which flows into the intra-room flow channel versus the proportion of air from the central HVAC which flows into ambient air in the bedroom. In an example, the one or more intra-room flow channels can comprise one or more modular sections. In an example, the one or more intra-room flow channels can comprise one or more modular sections selected from the group consisting of: a first type of intra-room flow channel modular section which spans straight along a wall; a second type of intra-room flow channel modular section which spans around a corner where two walls intersect; and a third type of intra-room flow channel modular section which at least partially covers a central HVAC vent.

In an example, the bedding component can be selected from the group consisting of: bed mattress, bed mattress pad or topper, lower/fitted bed sheet, upper bed sheet, blanket, comforter, quilt, and other specialized bedding. In an example, the one or more intra-bed flow channels can span at least a portion of the perimeter of the bed. In an example, the one or more intra-bed flow channels can span a right side, a left side, and a foot side of the perimeter of the bed. In an example, the bedding component can have longitudinal fabric channels along at least a portion of the perimeter of the bed into which one or more intra-bed flow channels are inserted. In an example, the bedding component can be a lower/fitted bed sheet with longitudinal fabric channels around its right side, its left side, and its foot side and wherein one or more intra-bed flow channels are inserted into the longitudinal fabric channels.

In an example, the device or system can further comprise a plurality of valves which are in fluid communication with the one or more intra-bed flow channels, wherein the valves are manually opened or closed by turning a knob, pivoting a lever, rotating a threaded member, sliding a strip, or placing a cap. In an example, the device or system can further comprise a plurality of valves in fluid communication with the one or more intra-bed flow channels, wherein the valves are automatically opened or closed by an electric actuator, hydraulic actuator, or pneumatic actuator.

In an example, the device or system can further comprise one or more sensors selected from the group consisting of: temperature sensor, pressure sensor, force sensor, moisture sensor, humidity sensor, motion sensor, sound sensor, light sensor, optical sensor, infrared sensor, spectroscopic sensor, electromagnetic energy sensor, EEG sensor, and EMG sensor.

In an example, a device or system to control the climate of one or more areas of a bed can comprise: a first intra-bed flow channel, wherein the first intra-bed flow channel conducts a liquid through a bedding component; and a second intra-bed flow channel, wherein the second intra-bed flow channel conducts air through the bedding component, and wherein the first intra-bed flow channel is inside the second intra-bed flow channel. In an example, there can be holes and/or openings in the second intra-bed flow channel through which air escapes.

In an example, a leak reduction mechanism for a device or system which controls the climate of one or more areas of a bed can comprise: an intra-bed flow channel which conducts a liquid through a bedding component; one or more valves which are in fluid communication with the intra-bed flow channel; one or more leak detection sensors selected from the group consisting of (a) a moisture and/or humidity sensor under the section of the intra-bed flow channel, (b) a pressure sensor which is in fluid communication with the section of the intra-bed flow channel, and (c) a fluid volume sensor which is in fluid communication with the section of the intra-bed flow channel; wherein the one or more valves are automatically closed if data from the one or more sensors indicates liquid leaking from the section. In an example, there can be a first valve at a first end of the section and a second valve at a second end of the section, wherein both valves are closed if data from the one or more sensors indicates liquid leaking from the section.

In an example, a device or system to change the temperature and/or moisture level of a bed can comprise: a fitted sheet or mattress pad which is configured to lie over a bed mattress and under a person while they sleep; one or more longitudinal channels which are made from fabric and span at least a portion of the perimeter of the sheet or mattress pad; one or more longitudinal air conduits which are removably-inserted into the longitudinal channels, wherein there are openings and/or holes in the air conduits through which air flows out of the air conduits into the bed to change the temperature and/or moisture level of the bed; and a flow accelerator which accelerates a flow of air into the longitudinal air conduits.

In an example, the one or more longitudinal channels on the perimeter of the sheet or mattress pad can further comprise: a first channel along the upper right side of the sheet or mattress pad; a second channel along the upper left side of the sheet or mattress pad; and a third channel along the foot of the sheet or mattress pad. In an example, the one or more longitudinal air conduits on the perimeter of the sheet or mattress pad can further comprise: a first air conduit along the upper right side of the sheet or mattress pad; a second air conduit along the upper left side of the sheet or mattress pad; and a third air conduit along the foot of the sheet or mattress pad.

In an example, the one or more longitudinal air conduits on the perimeter of the sheet or mattress pad can further comprise: a first air conduit which is inserted into a first fabric channel along the upper right side of the sheet or mattress pad; a second air conduit which is inserted into a second fabric channel along the upper left side of the sheet or mattress pad; and a third air conduit which is inserted into a third fabric channel along the foot of the sheet or mattress pad.

Management of the temperature and moisture/humidity level of areas (e.g. zones) within a bed can help to improve a person's sleep quality, prevent bed ulcers, and reduce energy costs. People generally sleep better in bed at their preferred temperature settings. In an example, a device or system can control different temperature settings for different time intervals during the night and/or for different phases of sleep. In an example, it can be quicker and more energy-efficient to cool, heat, and/or dehumidify an area within a bed (e.g. the space between lower and upper bed sheets) directly, instead of doing so indirectly by cooling, heating, or dehumidifying an entire bedroom.

Heat generation be done within a bedroom or even within bedding itself. For example, electrical energy can be easily converted into thermal energy which is used to heat a bed. Cooling a bed can be more challenging. Generally cooling is done by extracting thermal energy from air or liquid which is circulated through a bed. However, the extracted thermal energy has to go somewhere. One option to disperse the thermal energy into ambient air in the bedroom. However, if heat accumulates in the bedroom over time, then this can erode the effectiveness of bed cooling. In an example, air circulation through a bed space can help to remove moisture which could otherwise cause bed ulcers. However, moisture extracted from a bed also has to go somewhere and moisture accumulation in ambient bedroom air can also cause problems.

To address these challenges, a device or system can include one or more intra-room flow channels which provide isolated airflow between a bed and a central HVAC (Heating Ventilation and Air Conditioning) system for a building. This can help to completely remove thermal energy and/or moisture from a bedroom for more effective bed cooling and dehumidification. In an example, one or more intra-room flow channels can conduct air from a HVAC vent to a bed space and/or air from a bed to a HVAC vent. Disclosed herein are devices or systems which change, manage, and/or control the climate (e.g. temperature and/or humidity) of one or more spaces and/or areas (e.g. zones) within a bed, including devices and systems which create fluid communication between those spaces and/or zones and a central HVAC system for a building.

In an example, a device or system to change, manage, and/or control the climate of one or more areas (e.g. zones) of a bed can comprise: one or more intra-room flow channels which conduct a flowable substance between a first component and a second component in bedroom (e.g. room containing a bed), wherein the first component is (a) an airflow vent (e.g. on a room floor, wall, or ceiling) though which air enters the room from a central HVAC system, (b) an airflow vent (e.g. on a room floor, wall, or ceiling) through which air exits the room to a central HVAC system, or (c) a window-mounted air conditioner, and wherein the second component is a bedding component (e.g. a bed mattress, mattress pad or topper, lower and/or fitted bed sheet, upper bed sheet, blanket, comforter, quilt, or other specialized bedding); and one or more intra-bed flow channels which conduct the flowable substance through the bedding component in order to change the climate (e.g. temperature and/or humidity) of one or more areas (e.g. zones) of a bed.

In an example, a first component can be a vent (e.g. an air register) through which air enters a room from a central HVAC (heating ventilation and cooling) system. In another example, a first component can be an interior portion of a window air conditioner. In an example, a second component can be a bed or article of bedding. In an example, an intra-room flow channel can provide isolated airflow between a central HVAC system or window air conditioner. In an example, an intra-room flow channel which isolates fluid communication between a bed and a central HVAC vent (or window air conditioner) can increase the cooling or heating efficiency of a device or system which controls the climate of space within the bed.

In an example, an intra-room flow channel can conduct cooled or heated air to a bed (or bedding component) from a vent through which air enters a bedroom from a central HVAC system. In an example, this cooled or heated air can directly cool or heat a space within the bed without the need for a separate thermal component (e.g. heating or cooling unit) in the device. In another example, this air can be further cooled or heated by two separate thermal components (e.g. heating or cooling units) in the device, one component for each zone (e.g. each side) of a bed. In an example, an intra-room flow channel can conduct warm or humid air out from a bed (e.g. from between lower and upper sheets) to a vent wherein this air exits a bedroom to a central HVAC system.

In an example, a device or system can draw ambient air from a bedroom into a bed (e.g. into a space between lower and upper sheets), wherein this air is later drawn into a central HVAC system through an intra-room flow channel. In an example, an inflow of cool or warm air from a first flow channel can directly cool or heat a space within the bed without the need for a separate thermal component (e.g. heating or cooling unit) in the device. In an example, an outflow of warm or humid air through the second flow channel can avoid accumulation of unwanted heat (e.g. if the bed is being cooled) or humidity in the bedroom.

In an example, a wall-based flow channel can be attached to a wall. In an example, a wall-based flow channel can have a flat (vertical) side which is placed against a wall. In an example, a wall-based flow channel can have a height which is greater than its width. In an example, a wall-based flow channel can protrude from a wall by less than 2 inches. In an example, a wall-based flow channel can protrude from a wall by between 2 and 6 inches. In an example, a wall-based flow channel can be placed along a lower portion of one or more walls except for a vertical columnar section to reach a vent which is high up on a wall.

In an example, an intra-room flow channel (between a bed and an airflow vent) can be substantially co-planar with (e.g. flat against) a bedroom floor, except for a vertical portion if a vent is on a bedroom wall. In example, a first type of modular flow section can be a straight modular flow section along a wall. In example, a second type of modular flow section can be a corner modular flow section which goes around a room corner. In example, a third type of modular flow section can be a vent modular flow section which covers am HVAC vent.

In an example, a wall-based flow channel can have planar (e.g. flat) vertical sides and a sloped top surface, wherein the top surface slopes away from the wall. In an example, a wall-based flow channel can be modular. In an example, a first type of wall-based channel module can be straight to span a straight section of a wall. In an example, a second type of wall-based channel module can be angled (e.g. right angled) to span a room corner between two walls. In an example, a wall-based flow channel can be articulated, pleated, and/or telescoping. In an example, a wall-based flow channel can have one or more articulated, pleated, and/or telescoping sections which can be flexed, expanded, and/or extended. In an example, a wall-based flow channel can have one or more articulated, pleated, and/or telescoping wall-based flow modules.

In an example, the side of a wall-based flow channel which faces away from a wall can have a wood-grain appearance. In an example, a wall-based flow channel can be available in different colors to match the color of a wall or be an accent color for the wall. In an example, a floor-based flow channel can have a flat bottom which is placed against a floor. In an example, a floor-based flow channel can have a width which is more than three times its height. In an example, a floor-based flow channel can protrude from a floor by between 1 and 3 inches.

In an example, a floor-based flow channel can have a cross-sectional shape which is selected from the group consisting of: rectangle with one rounded (e.g. quarter-circle arc) end, rectangle with one rounded (e.g. quarter-circle arc) vertex, rectangular, rectangular with rounded vertexes, straight lower and lateral perimeter sides and arcuate upper perimeter side, circular, convex, elliptical, keystone, oval, polygonal, rounded-polygonal, square, and trapezoidal. In an example, a floor-based flow channel can have planar (e.g. flat) horizontal sides and an arcuate side surface, wherein the side surface has a conic section shape.

In an example, a floor-based flow channel can comprise a plurality of floor-based channel modules which are connected together to span between a bed and a vent. In an example, a second type of floor-based channel module can be angled (e.g. right angled) to span a room corner between two walls. In an example, a portion of an intra-room flow channel can be shaped and/or sized to be inserted into a central HVAC vent (e.g. air register) to enable isolated air flow between the vent and the intra-room flow channel. In an example, an intra-room flow channel can be modular, wherein one channel module is shaped and/or sized to cover, connect with, or be inserted into a central HVAC vent (e.g. air register) to enable fluid communication between air flowing through the vent and air flowing through channel.

In an example, a portion of an intra-room flow channel can be shaped and/or sized to fit onto a window air conditioner to enable isolated air flow between the air conditioner and the intra-room flow channel. In an example, an intra-room flow channel can be modular, wherein one channel module is shaped and/or sized to cover, connect with, or fit onto a window air conditioner to enable fluid communication between air flowing through the air conditioner and air flowing through channel.

In an example, an intra-room flow channel can connect with a central HVAC vent (e.g. air register) to enable fluid communication between air flowing through the vent and air flowing through channel. In an example, an intra-room flow channel can include an opening which is placed over a central HVAC vent (e.g. air register) to enable fluid communication between air flowing through the vent and air flowing through channel. In an example, an intra-room flow channel can comprise a plurality of channel modules which are connected together to span between a bed and a vent, wherein a first type of channel module is straight to span a straight section of a wall, a second type of channel module is angled (e.g. right angled) to span a room corner between two walls, and a third type of channel module is shaped and/or sized to cover, connect with, or be inserted into a central HVAC vent (e.g. air register).

In an example, a section and/or module can further comprise an air valve which changes the amount of air flowing out from the vent which enters the bedroom vs. an intra-room channel. In an example, an air valve can change the proportion of air coming out from the vent which flows into the bedroom vs. an intra-room channel. In an example, this air valve can have a first configuration in which more air flows from the vent into the bedroom than flows into the intra-room flow channel and a second configuration in which more air flows from the vent into the intra-room flow channel than flows into the bedroom.

In an example, this air valve can be moved automatically by an (electromagnetic) actuator from the first configuration to the second configuration, wherein the actuator is controlled by a data processor in the device or system. In an example, this air valve can be moved automatically based on data from temperature and/or humidity sensors in a bed. In an example, a floor-based flow channel can have one or more articulated, pleated, and/or telescoping sections. In an example, a floor-based flow channel can have one or more articulated, pleated, and/or telescoping sections whose lengths can be changed. In an example, the side of a floor-based flow channel which faces away from a floor can have a metallic color. In an example, a floor-based flow channel can be transparent or reflective to blend in with room decor.

In an example, a bedding component can be a bed mattress, mattress pad or topper, lower and/or fitted bed sheet, upper bed sheet, blanket, comforter, quilt, or other specialized bedding. In an example, a bedding component (e.g. bed mattress, mattress pad or topper, lower and/or fitted bed sheet, upper bed sheet, blanket, comforter, quilt, or other specialized bedding) can further comprise intra-bed flow channels which span a bed in a longitudinal (e.g. head to foot, or vice versa) manner. In an example, a bedding component (e.g. bed mattress, mattress pad or topper, lower and/or fitted bed sheet, upper bed sheet, blanket, comforter, quilt, or other specialized bedding) can further comprise a plurality of undulating (e.g. sinusoidal) intra-bed flow channels. In an example, a bedding component (e.g. bed mattress, mattress pad or topper, lower and/or fitted bed sheet, upper bed sheet, blanket, comforter, quilt, or other specialized bedding) can further comprise a plurality of nested intra-bed flow channels.

In an example, a flowable substance can be air. In an example, an intra-room flow channel can conduct air from an HVAC vent to bedding component (e.g. a bed mattress, mattress pad or topper, lower and/or fitted bed sheet, upper bed sheet, blanket, comforter, quilt, or other specialized bedding). In an example, an intra-room flow channel can conduct air from a bedding component to an HVAC vent. In an example, an intra-room flow channel can conduct air from a space in a bed (e.g. the space between lower and upper sheets) to a HVAC vent.

In an example, an intra-room flow channel can conduct air through a bedding component (e.g. a bed mattress, mattress pad or topper, lower and/or fitted bed sheet, upper bed sheet, blanket, comforter, quilt, or other specialized bedding) into a space between lower and upper sheets in a bed. In an example, there can be a right-side airflow loop (e.g. fluid circuit or zone) for the right side of the bed and a left-side airflow loop (e.g. fluid circuit or zone) for the left side of the bed, wherein airflow temperatures and flow rates for these two loops can be independently adjusted.

In an example, an intra-room flow channel can conduct a liquid between a bed and a HVAC vent. In an example, an intra-room flow channel can conduct a cooled or warmed liquid between a bed and a HVAC vent. In an example, an intra-room flow channel can conduct a cooled or warmed liquid from a HVAC vent to a bed. In an example, an intra-room flow channel can conduct a liquid between a bed and a heat exchanger and/or fluid flow coil which is within one foot of a HVAC vent. In an example, an intra-room flow channel can conduct a liquid between a bedding component and a HVAC vent. In an example, an intra-room flow channel can conduct a cooled or warmed liquid from a bedding component to a HVAC vent. In an example, an intra-room flow channel can conduct a liquid between a heat exchanger and/or fluid flow coil in a bedding component and a HVAC vent.

In an example, a device or system to change, manage, and/or control the climate of one or more areas of a bed can comprise an intra-room flow channel which conducts air between a bed and a central HVAC vent and an intra-bed flow channel which conducts air through a bedding component (e.g. a bed mattress, mattress pad or topper, lower and/or fitted bedding component sheet, upper bedding component sheet, blanket, comforter, quilt, or other specialized bedding). In an example, a device or system which changes, manages, and/or controls the climate (e.g. temperature and/or humidity) of one or more areas (e.g. sides or zones) of a bed can use both air and a liquid (e.g. water).

In an example, a device or system to change, manage, and/or control the climate of one or more areas of a bed can comprise an intra-room flow channel which conducts liquid between a bed and a central HVAC vent and an intra-bed flow channel which conducts liquid through a bedding component (e.g. a bed mattress, mattress pad or topper, lower and/or fitted bedding component sheet, upper bedding component sheet, blanket, comforter, quilt, or other specialized bedding). In an example, a device or system can further comprise two intra-room flow channels which conduct flows of a liquid (e.g. water) in two directions, respectively, between a near-vent component (e.g. within two feet of a central HVAC vent) and a near-bed component (e.g. within two feet of a bed). In an example, flow channels which direct flows in opposite directions (e.g. inflow and outflow) can be contained in a single channel housing.

In an example, a device or system to change, manage, and/or control the climate of one or more areas (e.g. zones) of a bed can comprise one or more intra-bed flow channels which conduct a flowable substance through a bed in order to change the climate (e.g. temperature and/or humidity) of a space between sheets around a sleeping person. In an example, one or more intra-bed flow channels can be located on the side sections of the perimeter of a bed. In an example, one or more intra-bed flow channels can be located on the side and foot sections of the perimeter of a bed.

In an example, a device or system to change, manage, and/or control the climate of one or more areas (e.g. zones) of a bed can comprise one or more intra-bed flow channels which conduct a flowable substance through a bedding component (e.g. a bed mattress, mattress pad or topper, lower and/or fitted bed sheet, upper bed sheet, blanket, comforter, quilt, or other specialized bedding). In an example, one or more intra-bed flow channels can be located on the side sections of the perimeter of a bedding component. In an example, one or more intra-bed flow channels can be located on the side and foot sections of the perimeter of a bedding component.

In an example, a device or system to change, manage, and/or control the climate of one or more areas (e.g. zones) of a bed can comprise one or more airflow channels which conduct air through a bed in order to change the climate (e.g. temperature and/or humidity) of a space between sheets around a sleeping person. In an example, one or more airflow channels can be located on the side sections of the perimeter of a bed. In an example, one or more airflow channels can be located on the side and foot sections of the perimeter of a bed.

In an example, a device or system to change, manage, and/or control the climate of one or more areas (e.g. zones) of a bed can comprise one or more airflow channels which conduct air through a bedding component (e.g. a bed mattress, mattress pad or topper, lower and/or fitted bed sheet, upper bed sheet, blanket, comforter, quilt, or other specialized bedding). In an example, one or more airflow channels can be located on the side sections of the perimeter of a bedding component. In an example, one or more airflow channels can be located on the side and foot sections of the perimeter of a bedding component.

In an example, a device or system to change, manage, and/or control the climate of one or more areas (e.g. zones) of a bed can comprise one or more liquid flow channels which conduct a liquid through a bed in order to change the climate (e.g. temperature and/or humidity) of a space between sheets around a sleeping person. In an example, one or more liquid flow channels can be located on the side sections of the perimeter of a bed. In an example, one or more liquid flow channels can be located on the side and foot sections of the perimeter of a bed.

In an example, a device or system to change, manage, and/or control the climate of one or more areas (e.g. zones) of a bed can comprise one or more liquid flow channels which conduct a liquid through a bedding component (e.g. a bed mattress, mattress pad or topper, lower and/or fitted bed sheet, upper bed sheet, blanket, comforter, quilt, or other specialized bedding). In an example, one or more liquid flow channels can be located on the side sections of the perimeter of a bedding component. In an example, one or more liquid flow channels can be located on the side and foot sections of the perimeter of a bedding component.

In an example, there can be a single intra-bed flow channel (e.g. flow loop or circuit) which conducts a flowable substance around (a portion of) the perimeter of a bed or bedding component. In an example, there can be: a first intra-bed flow channel (e.g. flow loop or circuit) which conducts a flowable substance through a first side (e.g. right side) of a bed or bedding component; and a second intra-bed flow channel which conducts the flowable substance through a second side (e.g. left side) of the bed. In an example, there can be: a first intra-bed flow channel (e.g. flow loop or circuit) which conducts a flowable substance through a first side (e.g. right side) section of the perimeter of a bed or bedding component; and a second intra-bed flow channel which conducts the flowable substance through a second side (e.g. left side) section of the perimeter of the bed.

In an example, there can be a single intra-bed flow channel (e.g. flow loop or circuit) which directs a flowable substance toward the center (e.g. central longitudinal axis) of a bed from the perimeter of a bed or bedding component. In an example, there can be: a first intra-bed flow channel (e.g. flow loop or circuit) which directs a flowable substance toward the center (e.g. central longitudinal axis) of a bed from a first side (e.g. right side) of the bed or bedding component; and a second intra-bed flow channel which directs the flowable substance toward the center (e.g. central longitudinal axis) of the bed from a second side (e.g. left side) of the bed.

In an example, there can be: a first intra-bed flow channel (e.g. flow loop or circuit) which directs a flowable substance toward the center (e.g. central longitudinal axis) of a bed from a first side (e.g. right side) section of the perimeter of a bed or bedding component; and a second intra-bed flow channel which directs the flowable substance toward the center (e.g. central longitudinal axis) of the bed from a second side (e.g. left side) section of the perimeter of the bed. In an example, an intra-bed flow channel can conduct airflow through a bedding component to change, manage, and/or control the climate (e.g. temperature and/or humidity) of a space between a bottom sheet and an upper sheet.

In an example, a device or system can further comprise a plurality of valves which adjust airflows through the plurality of holes. In another example, each individually-adjustable valve can control airflow through several holes. In an example, the inner layer can be made from a (flexible and non-air-permeable) polymer and the outer layer can be made from (a soft and air-permeable) fabric. In an example, an intra-bed flow channel can be longitudinally undulating and/or sinusoidal.

In an example, an air-conducting intra-bed flow channel can be inserted into longitudinal channels on (a portion of) the perimeter of a bedding component (e.g. bed mattress, mattress pad or topper, bottom sheet, upper sheet, or blanket). In an example, an air-conducting intra-bed flow channel can comprise and air-conducting tube which is inserted (e.g. slid) into longitudinal channels or channels on the perimeter of a bedding component (e.g. bed mattress, mattress pad or topper, bottom sheet, upper sheet, or blanket). In an example, one or more air-conducting intra-bed flow channels can be removed from a bedding component before the bedding component is washed and then re-inserted into the bedding component after the bedding component has been washed.

In an example, a device or system to change the temperature and/or moisture level of a bed can comprise: a fitted sheet or mattress pad which is configured to lie over a bed mattress and under a person while they sleep; one or more longitudinal channels which are made from fabric and span at least a portion of the perimeter of the sheet or mattress pad; one or more longitudinal air conduits which are removably-inserted into the longitudinal channels, wherein there are openings and/or holes in the air conduits through which air flows out of the air conduits into the bed to change the temperature and/or moisture level of the bed; and a flow accelerator which accelerates a flow of air into the longitudinal air conduits.

In an example, the one or more longitudinal channels on the perimeter of the sheet or mattress pad can further comprise: a first channel along the upper right side of the sheet or mattress pad; a second channel along the upper left side of the sheet or mattress pad; and a third channel along the foot of the sheet or mattress pad. In an example, the one or more longitudinal air conduits on the perimeter of the sheet or mattress pad can further comprise: a first air conduit along the upper right side of the sheet or mattress pad; a second air conduit along the upper left side of the sheet or mattress pad; and a third air conduit along the foot of the sheet or mattress pad.

In an example, the one or more longitudinal air conduits on the perimeter of the sheet or mattress pad can further comprise: a first air conduit which is inserted into a first fabric channel along the upper right side of the sheet or mattress pad; a second air conduit which is inserted into a second fabric channel along the upper left side of the sheet or mattress pad; and a third air conduit which is inserted into a third fabric channel along the foot of the sheet or mattress pad.

In an example, such a device or system can draw ambient air from the bedroom instead of through an isolated conduit to a central HVAC vent and send this air through intra-bed flow channels. In an example, such a device or system can cool or heat ambient air from the bedroom and send this air through intra-bed flow channels. In an example, a device or system to change, manage, and/or control the climate of one or more areas (e.g. zones) of a bed can further comprise a plurality of valves which change flow rates of a flowable substance to different areas (e.g. zones) of a bed. This enables one or more people to adjust fluid flow (e.g. airflow or liquid flow) to different areas (e.g. zones) of a bed.

In an example, there can be one valve for each hole (or opening) or flow loop in an intra-bed flow channel. In an example, individual valves can be individually and independently opened or closed in order to increase or decrease fluid flow (e.g. airflow or liquid flow) to different areas (e.g. zones) of a bed. In an example, valves can be manually opened or closed by turning a knob, pivoting a lever, rotating a threaded member, sliding a strip, placing a cap, or other manual mechanism. In an example, valves can be automatically opened or closed by an electric actuator, hydraulic actuator, or pneumatic actuator.

In an example, different valves around the perimeter of a bed can be individually-adjusted to change the amounts of airflow to different areas (e.g. sides or zones) of a bed. It also allows two people on different sides of a bed to receive more or less airflow on different portions of their bodies, according to their different preferences. In an example, there can be between 5 and 10 valves on each lateral (e.g. right and left) side of a bed or bedding component, wherein these valves adjust the flow of a flowable substance through the bedding component and/or into a space in the bed. In an example, there can be a plurality of on each lateral (e.g. right and left) side of a bed or bedding component, wherein these valves adjust the flows of air from intra-bed flow channels into different areas (e.g. sides or zones) of the bed.

In an example, a flow-accelerating component can be a blower, fan, turbine, impeller, or pump. In an example, a device or system can include a flow-accelerating component (e.g. a blower, fan, turbine, or impeller) which is near (e.g. within two feet of) a bed. In an example, a device or system to change, manage, and/or control the climate of one or more areas (e.g. zones) of a bed can further comprise one or more flow-accelerating component. In an example, there can be a flow-accelerating component in fluid communication with an intra-room flow channel. In an example, there can be a flow-accelerating component near (e.g. within one foot of) an HVAC vent. In an example, there can be a flow-accelerating component near (e.g. within one foot of) a bedding component. In an example, there can be a flow-accelerating component in a housing under a bed.

In an example, a device or system can comprise two or more flow-accelerating components which accelerate the flow rates of a flowable substance through two or more intra-room flow channels, respectively. In an example, a device or system can comprise two or more flow-accelerating components which accelerate flows of a flowable substance through different areas (e.g. zones) of a bed. In an example, a device or system to change, manage, and/or control the climate of one or more areas (e.g. zones) of a bed can further comprise one or more cooling (or heating) components. In an example, a cooling component can comprise a heat exchange unit. In an example, a cooling component can comprise a heat exchange unit.

In an example, a device or system can further comprise a heat exchange unit which transfers heat from air to a liquid, or vice versa. In an example, a heat exchange unit can be near (e.g. within two feet of) an HVAC vent. In an example, a device or system to change, manage, and/or control the climate of one or more areas (e.g. zones) of a bed can further comprise one or more heating (or cooling) components. In an example, a heating component can heat a flowable substance by converting electrical energy to thermal energy. In an example, a heating component can heat a flowable substance by extracting heat from air outside a bed and then transferring that heat to a flowable substance before it is conducted through an intra-bed flow channel.

In an example, there can be a cooling component in fluid communication with an intra-room flow channel. In an example, there can be a cooling component near (e.g. within one foot of) an HVAC vent. In an example, there can be a cooling component near (e.g. within one foot of) a bedding component. In an example, there can be a cooling component in a housing under a bed. In an example, there can be a heating component in fluid communication with an intra-room flow channel. In an example, there can be a heating component near (e.g. within one foot of) an HVAC vent. In an example, there can be a heating component near (e.g. within one foot of) a bedding component. In an example, there can be a heating component in a housing under a bed.

In an example, a device or system can comprise two or more cooling components which decrease the temperatures of a flowable substance through two or more intra-room flow channels, respectively. In an example, a device or system can comprise two or more cooling components which decrease the temperatures of a flowable substance which flows through different areas (e.g. zones) of a bed. In an example, a device or system can comprise two or more heating components which increase the temperatures of a flowable substance through two or more intra-room flow channels, respectively. In an example, a device or system can comprise two or more heating components which increase the temperatures of a flowable substance which flows through different areas (e.g. zones) of a bed.

In an example, a device or system which changes, manages, and/or controls the climate of a bed using a liquid flowable substance can further comprise a leak mitigation mechanism. In an example, a device or system can further comprise one or more moisture and/or humidity sensors, wherein the system stops the flow of a liquid flowable substance through a selected fluid flow loop or circuit if the sensors detect abnormally low fluid pressure or quantity concerning (e.g. near) that loop or circuit. In an example, a leak mitigation mechanism can comprise stopping the flow of a flowable substance through an intra-bed flow channel if an abnormal (e.g. high) amount of moisture and/or humidity is detected in a bed. In an example, a leak mitigation mechanism can comprise stopping the flow of a flowable substance through an intra-bed flow channel if an abnormal (e.g. low) quantity of the liquid flowable substance is detected.

In an example, a device or system which changes, manages, and/or controls the climate of a bed using a liquid flowable substance can further comprise a leak mitigation mechanism. In an example, a device or system can further comprise one or more moisture and/or humidity sensors, wherein the system stops the flow of a liquid flowable substance through a selected fluid flow loop or circuit if the sensors detect abnormally rapid decreases in fluid pressure or quantity concerning (e.g. near) that loop or circuit. In an example, a leak mitigation mechanism can comprise stopping the flow of a flowable substance through an intra-bed flow channel if an abnormal (e.g. rapid) increase in moisture and/or humidity is detected in a bed. In an example, a leak mitigation mechanism can comprise stopping the flow of a flowable substance through an intra-bed flow channel if an abnormal (e.g. rapid) decrease in the quantity of the liquid flowable substance is detected.

In an example, a device or system which changes, manages, and/or controls the climate of a bed using a liquid flowable substance can further comprise a leak mitigation mechanism. In an example, a device or system can further comprise one or more moisture and/or humidity sensors, wherein the system suctions a liquid flowable substance out from a selected fluid flow loop or circuit if the sensors detect abnormally low fluid pressure or quantity concerning (e.g. near) that loop or circuit. In an example, a leak mitigation mechanism can comprise suctioning a flowable substance out from an intra-bed flow channel if an abnormal (e.g. high) amount of moisture and/or humidity is detected in a bed. In an example, a leak mitigation mechanism can comprise suctioning a flowable substance out from an intra-bed flow channel if an abnormal (e.g. low) quantity of the liquid flowable substance is detected. In an example, a leak mitigation mechanism can comprise suctioning a flowable substance out from an intra-bed flow channel if an abnormal (e.g. low) pressure of the liquid flowable substance is detected.

In an example, a device or system can further comprise one or more moisture and/or humidity sensors, wherein the system suctions a liquid flowable substance out from a selected fluid flow loop or circuit if the sensors detect abnormally rapid increases in moisture and/or humidity concerning (e.g. near) that loop or circuit. In an example, a leak mitigation mechanism can comprise suctioning a flowable substance out from an intra-bed flow channel if an abnormal (e.g. rapid) increase in moisture and/or humidity is detected near the intra-bed flow channels. In an example, a leak mitigation mechanism can comprise suctioning a flowable substance out from an intra-bed flow channel if an abnormal (e.g. rapid) increase in moisture and/or humidity is detected in the intra-room flow channels. In an example, a leak mitigation mechanism can comprise suctioning a flowable substance out from an intra-bed flow channel if an abnormal (e.g. rapid) decrease in the pressure of the liquid flowable substance is detected.

In an example, there can be a plurality of automatic valves which define different sections along one or more intra-bed flow channels, wherein valves on either side of a section automatically close if a leak is detected in that section. In an example, a device or system can comprise one or more pressure sensors and/or moisture sensors, wherein detection of a leak in a particular section triggers closure of valves to isolate that section from the rest of the intra-bed flow channels and mitigate leakage. In an example, this device or system can further comprise a local dehumidifying component which is located in the bedroom. In an example, this device or system can further comprise a dehumidifying component which extracts water from air from a bed (e.g. out from the space between bottom and upper sheets). In an example, water extracted from this air can be temporarily stored in a chamber (e.g. reservoir) which is emptied later.

In an example, the amount of flowable substance in this reservoir can be monitored and the device or system can send an alert if this amount drops below a selected quantity and/or level (e.g. due to evaporation or leakage). In an example, this device or system can further comprise sensors which monitor characteristics of the flowable substance in this reservoir, wherein these characteristics are selected from the group consisting of: quantity, temperature, microbial growth, concentration, pressure, clarity, and purity. In an example, the device or system can automatically dispense disinfectant into a liquid flowable substance when microbial growth is detected in the liquid flowable substance.

In an example, a device or system which changes, manages, and/or controls the climate of one or more areas (e.g. zones) of a bed using a liquid flowable substance can further comprise a wireless remote control device. In an example, a wireless remote control device can have a soft (e.g. compressible fabric) housing like a plush toy (e.g. plush animal or ball). In an example, a wireless remote control device can be a (soft) hand-held device with a function (e.g. control of heating or cooling or airflow rate) which is activated by compression, wherein a person in bed can increase or decrease heating or cooling of airflow through the bed by compressing (or releasing) the device.

In an example, a wireless remote control device can be a (soft) hand-held device with a function (e.g. control of heating or cooling or airflow rate) which is activated by compression, wherein a person in bed can increase or decrease heating or cooling of a flowable substance through the bed by compressing (or releasing) the device. In an example, a system can further comprise a wearable item (e.g. smart watch, smart ring, headband, or smart clothing) with biometric sensors, wherein this item is in wireless communication with other components in the system. In an example, one or more sensors selected from the group consisting of: temperature sensor, pressure sensor, force sensor, moisture sensor, humidity sensor, motion sensor, sound sensor, light sensor, optical sensor, infrared sensor, spectroscopic sensor, electromagnetic energy sensor, EEG sensor, and EMG sensor.

In an example, this device or system can further comprise a wireless data transmitter. In an example, one or more sensors in a bed area (e.g. zone) can be selected from the group consisting of: temperature sensor, pressure sensor, force sensor, moisture sensor, humidity sensor, motion sensor, sound sensor, light sensor, optical sensor, infrared sensor, spectroscopic sensor, electromagnetic energy sensor, EEG sensor, and EMG sensor. This invention is a device or system which changes, manages, and/or controls the climate (e.g. temperature and/or humidity) of one or more spaces and/or areas (e.g. zones) within a bed. For example, people sleep better in bed at their preferred temperature setting. In another example, air circulation through a bed space can help to remove moisture and warm spots which could otherwise cause bed ulcers.

In an example, one can generate heat to warm a bed by converting electrical energy into thermal energy (e.g. with electrical coils). However, cooling or dehumidifying a bed is more challenging because heat or moisture which is extracted from a bed space has to go somewhere. However, if heat or humidity accumulates in the bedroom, then this can make it difficult to cool or dehumidify the bed. This can extract heat or moisture from a bedroom as well as a bed. An intra-room flow channel conducts a flowable substance between a first component in a bedroom and a second component in the bedroom. The vent can be on a bedroom floor, wall, or ceiling. In another example, a first component can be a vent (e.g. an air register) through which air exits a room to a central HVAC (Heating Ventilation And Cooling) system. In an example, a second component can be selected from the group consisting of: bed mattress; mattress pad or topper; lower and/or fitted bed sheet; upper bed sheet; blanket, comforter, or quilt; or other specialized bedding layer under or over a sleeping person.

In an example, isolated airflow with a HVAC system or window air conditioner can change the climate (e.g. temperature and/or humidity) in a bed space (e.g. between lower and upper sheets) more quickly and efficiently than is possible by using ambient air in a bedroom. For example, if air from a central HVAC system is sufficiently cool or warm, then it can be possible to cool or warm a space within a bed via an intra-room flow channel without the need for a separate thermal exchange (e.g. heating or cooling) unit as part of the device. An intra-room flow channel can send this heat out of the room via an outflow channel.

In an example, an intra-room flow channel can be insulated from ambient air in a bedroom. In another example, this air can be further cooled or heated by a separate thermal component (e.g. heating or cooling unit) in the device. In an example, a device or system can draw cooled or heated air from a central HVAC system through an intra-room flow channel into a bed, wherein this air flows around a sleeping person and then exits the bed into the ambient air of the bedroom. This can help to extract excessive heat or moisture from a bed and avoid having the heat or moisture build up in ambient air in the bedroom. In an example, a device or system can comprise two intra-room flow channels: a first flow channel which conducts (heated or cooled) air from an inflow vent from a central HVAC system to a bed; and a second flow channel which conducts (warm or humid) air from the bed to an outflow vent to the central HVAC system. Alternatively, this inflow of air can be further cooled or heated by one or more a separate thermal components (e.g. heating or cooling units) in the device.

In an example, an intra-room flow channel can a wall-based flow channel which spans along one or more walls between a bed and an HVAC vent. In preferred example, a wall-based flow channel can be along a wall, but not attached to the wall. In an example, a wall-based flow channel can be placed along a lower portion of one or more walls, below the height of electrical outlets (e.g. no more than one foot above the floor). In an example, a wall-based flow channel can have a height which is more than twice its width. In an example, a wall-based flow channel can protrude from a wall by between 1 and 3 inches. In an example, a wall-based flow channel can have a (vertical) height between 6 and 12 inches and a (horizontal) width between 1 and 6 inches. In an example, an intra-room flow channel (between a bed and an airflow vent) can be substantially co-planar with (e.g. flat against) one or more bedroom walls. In an example, an intra-room flow channel (between a bed and an airflow vent) can be comprised of connected modular flow sections.

In an example, a wall-based flow channel can have a cross-sectional shape which is selected from the group consisting of: rectangle with one rounded (e.g. quarter-circle arc) end, rectangle with one rounded (e.g. quarter-circle arc) vertex, rectangular, rectangular with rounded vertexes, straight lower and lateral perimeter sides and arcuate upper perimeter side, circular, convex, elliptical, keystone, oval, polygonal, rounded-polygonal, square, and trapezoidal. In an example, a wall-based flow channel can have planar (e.g. flat) vertical sides and an arcuate top surface, wherein the top surface has a conic section shape.

In an example, an wall-based flow channel can comprise a plurality of wall-based channel modules which are connected together to span between a bed and a vent. In an example, a first type of wall-based channel module can be straight to span a straight section of a wall horizontally. In an example, channel modules can be connected to each other by a connection mechanism selected from the group consisting of: snap, clip, clasp, clamp, hook, interlock, helical threads, strap, tape, adhesive, crimp, latch, magnet, hook and loop material, and pin. In an example, a wall-based flow channel can have one or more articulated, pleated, and/or telescoping sections. In an example, a wall-based flow channel can have one or more articulated, pleated, and/or telescoping sections whose lengths can be changed.

In an example, the side of a wall-based flow channel which faces away from a wall can have a metallic color. In an example, a wall-based flow channel can be transparent or reflective to blend in with room decor. In an example, an intra-room flow channel can a floor-based flow channel which spans along a room floor between a bed and an HVAC vent. In an example, a floor-based flow channel can have a width which is greater than its height. In an example, a floor-based flow channel can protrude from a floor by less than 2 inches. In an example, a floor-based flow channel can protrude from a floor by between 2 and 6 inches. In an example, a floor-based flow channel can have planar (e.g. flat) horizontal sides and a sloped side surface, wherein the side surface slopes away from the floor.

In an example, a floor-based flow channel can be modular. In an example, a first type of floor-based channel module can be straight to span a straight section of a wall. In an example, channel modules can be connected to each other by a connection mechanism selected from the group consisting of: snap, clip, clasp, clamp, hook, interlock, helical threads, strap, tape, adhesive, crimp, latch, magnet, hook and loop material, and pin. In an example, a portion of an intra-room flow channel can be shaped and/or sized to be inserted into a central HVAC vent (e.g. air register) to enable isolated air flow between the vent and the intra-room flow channel. In an example, an intra-room flow channel can be modular, wherein one channel module is shaped and/or sized to cover, connect with, or be inserted into a central HVAC vent (e.g. air register) to enable isolated air flow between the vent and the intra-room flow channel.

In an example, a portion of an intra-room flow channel can be shaped and/or sized to fit onto a window air conditioner to enable isolated air flow between the air conditioner and the intra-room flow channel. In an example, an intra-room flow channel can be modular, wherein one channel module is shaped and/or sized to cover, connect with, or fit onto a window air conditioner to enable isolated air flow between the air conditioner and the intra-room flow channel. In an example, an intra-room flow channel can connect with a central HVAC vent (e.g. air register) to enable isolated air flow between the vent and the intra-room flow channel. In an example, an intra-room flow channel can include an opening which is placed over a central HVAC vent (e.g. air register) to enable isolated air flow between the vent and the intra-room flow channel.

In an example, a section and/or module of an intra-room flow channel can be shaped and/or sized to cover, connect with, or be inserted into a central HVAC vent (e.g. air register). the amount of air flowing out from the vent which enters the intra-room flow channel. the intra-room flow channel. In an example, this air valve can be moved manually from the first configuration to the second configuration. In an example, this air valve can be moved automatically based on data from one or more sensors in a bed. In an example, a floor-based flow channel can be articulated, pleated, and/or telescoping. In an example, a floor-based flow channel can have one or more articulated, pleated, and/or telescoping sections which can be flexed, expanded, and/or extended. In an example, a floor-based flow channel can have one or more articulated, pleated, and/or telescoping floor-based flow modules. In an example, the side of a floor-based flow channel which faces away from a floor can have a wood-grain appearance. In an example, a floor-based flow channel can be available in different colors to match the color of a floor or be an accent color for the floor.

In an example, a bedding component (e.g. bed mattress, mattress pad or topper, lower and/or fitted bed sheet, upper bed sheet, blanket, comforter, quilt, or other specialized bedding) can further comprise intra-bed flow channels which span a bed in a lateral (e.g. right to left, or vice versa) manner. In an example, a bedding component (e.g. bed mattress, mattress pad or topper, lower and/or fitted bed sheet, upper bed sheet, blanket, comforter, quilt, or other specialized bedding) can further comprise intra-bed flow channels which span a bed in lateral (e.g. right to left. or vice versa) and longitudinal (e.g. head to foot, or vice versa) directions.

In an example, a bedding component (e.g. bed mattress, mattress pad or topper, lower and/or fitted bed sheet, upper bed sheet, blanket, comforter, quilt, or other specialized bedding) can further comprise a plurality of undulating (e.g. sinusoidal) intra-bed flow channels whose peaks and troughs intersect each other. In an example, a bedding component (e.g. bed mattress, mattress pad or topper, lower and/or fitted bed sheet, upper bed sheet, blanket, comforter, quilt, or other specialized bedding) can further comprise a plurality of radial (e.g. hub and spoke or star-burst array) intra-bed flow channels.

In an example, an intra-room flow channel can conduct air between a bed and a HVAC vent. In an example, an intra-room flow channel can conduct air from an HVAC vent to a space in a bed (e.g. the space between lower and upper sheets). In an example, an intra-room flow channel can conduct air from a bedding component to an HVAC vent. In an example, an intra-room flow channel can conduct air through a bedding component (e.g. a bed mattress, mattress pad or topper, lower and/or fitted bed sheet, upper bed sheet, blanket, comforter, quilt, or other specialized bedding). In an example, an intra-room flow channel can conduct air through a bedding component (e.g. a bed mattress, mattress pad or topper, lower and/or fitted bed sheet, upper bed sheet, blanket, comforter, quilt, or other specialized bedding) out from a space between lower and upper sheets in a bed.

In an example, a flowable substance can be a liquid (e.g. water). In an example, an intra-room flow channel can conduct a liquid between a bed and a HVAC vent. In an example, an intra-room flow channel can conduct a cooled or warmed liquid from a bed to a HVAC vent. In an example, an intra-room flow channel can conduct a liquid between a heat exchanger and/or fluid flow coil which is within one foot of a bed and a HVAC vent. In an example, an intra-room flow channel can conduct a liquid between a bedding component (e.g. a bed mattress, mattress pad or topper, lower and/or fitted bedding component sheet, upper bedding component sheet, blanket, comforter, quilt, or other specialized bedding) and a HVAC vent. In an example, an intra-room flow channel can conduct a cooled or warmed liquid between a bedding component and a HVAC vent. In an example, an intra-room flow channel can conduct a cooled or warmed liquid from a HVAC vent to a bedding component. In an example, an intra-room flow channel can conduct a liquid between a bedding component and a heat exchanger and/or fluid flow coil which is within one foot of a HVAC vent.

In an example, a device or system can include air-conducting tubes which direct air into a space between lower and upper sheets in a bed and air-conducting tubes which withdraw air from this space. In an example, a flowable substance in flow channels in a bedding component can be air and a flowable substance in flow channels between a bed and a central HVAC vent can be a liquid (e.g. water). In an example, a device or system to change, manage, and/or control the climate of one or more areas of a bed can comprise an intra-room flow channel which conducts air between a bed and a central HVAC vent and an intra-bed flow channel which conducts a liquid through a bedding component (e.g. a bed mattress, mattress pad or topper, lower and/or fitted bedding component sheet, upper bedding component sheet, blanket, comforter, quilt, or other specialized bedding), wherein there is a heat exchange unit between the intra-room flow channel and the intra-bed channel which transfers heat from the air to the liquid, or vice versa.

In an example, a device or system to change, manage, and/or control the climate of one or more areas of a bed can comprise an intra-room flow channel which conducts liquid between a bed and a central HVAC vent and an intra-bed flow channel which conducts a liquid through a bedding component (e.g. a bed mattress, mattress pad or topper, lower and/or fitted bedding component sheet, upper bedding component sheet, blanket, comforter, quilt, or other specialized bedding), wherein there is a heat exchange unit between the intra-room flow channel and the intra-bed channel which transfers heat from the liquid to the air, or vice versa. In an example, flow channels which direct flows in opposite directions (e.g. inflow and outflow) can be contained in a single housing. In an example, flow channels which direct flows in opposite directions (e.g. inflow and outflow) can be insulated and configured in parallel through a single channel housing.

In an example, one or more intra-bed flow channels can be located around (a portion of) the perimeter of a bed. In an example, one or more intra-bed flow channels can be located on the foot section of the perimeter of a bed. In an example, one or more intra-bed flow channels can span between 40% and 80% of the perimeter of a bed. In an example, one or more intra-bed flow channels can be located around (a portion of) the perimeter of a bedding component. In an example, one or more intra-bed flow channels can be located on the foot section of the perimeter of a bedding component. In an example, one or more intra-bed flow channels can span between 40% and 80% of the perimeter of a bedding component. In an example, one or more airflow channels can be located around (a portion of) the perimeter of a bed. In an example, one or more airflow channels can be located on the foot section of the perimeter of a bed. In an example, one or more airflow channels can span between 40% and 80% of the perimeter of a bed.

In an example, one or more airflow channels can be located around (a portion of) the perimeter of a bedding component. In an example, one or more airflow channels can be located on the foot section of the perimeter of a bedding component. In an example, one or more airflow channels can span between 40% and 80% of the perimeter of a bedding component. In an example, one or more liquid flow channels can be located around (a portion of) the perimeter of a bed. In an example, one or more liquid flow channels can be located on the foot section of the perimeter of a bed. In an example, one or more liquid flow channels can span between 40% and 80% of the perimeter of a bed. In an example, one or more liquid flow channels can be located around (a portion of) the perimeter of a bedding component. In an example, one or more liquid flow channels can be located on the foot section of the perimeter of a bedding component. In an example, one or more liquid flow channels can span between 40% and 80% of the perimeter of a bedding component.

In an example, there can be a plurality of intra-bed flow channels (e.g. flow loops or circuits) which conduct a flowable substance around (portions of) the perimeter of a bed or bedding component. In an example, the temperatures and/or flow rates of these two flows can be independently controlled. In an example, there can be: a first intra-bed flow channel (e.g. flow loop or circuit) which conducts a flowable substance through a first side (e.g. right side) section of the perimeter of a bed or bedding component; a second intra-bed flow channel which conducts the flowable substance through a second side (e.g. left side) section of the perimeter of the bed; and a third intra-bed flow channel which conducts the flowable substance through a foot section of the perimeter of the bed.

In an example, there can be a plurality of intra-bed flow channels (e.g. flow loops or circuits) which direct a flowable substance toward the center (e.g. central longitudinal axis) of a bed from the perimeter of a bed or bedding component. In an example, the temperatures and/or flow rates of these two flows can be independently controlled. In an example, there can be: a first intra-bed flow channel (e.g. flow loop or circuit) which directs a flowable substance toward the center (e.g. central longitudinal axis) of a bed from a first side (e.g. right side) section of the perimeter of a bed or bedding component; a second intra-bed flow channel which directs the flowable substance toward the center (e.g. central longitudinal axis) of the bed from a second side (e.g. left side) section of the perimeter of the bed; and a third intra-bed flow channel which directs the flowable substance toward the center (e.g. central longitudinal axis) of the bed from a foot section of the perimeter of the bed.

In an example, an air-conducting intra-bed flow channel can be a flexible longitudinal tube which has a central air pathway and a plurality of holes in the pathway through which air flows out into a bed space. In an example, there can be an individually-adjustable valve for each hole in the tube. In an example, an air-conducting intra-bed flow channel can comprise a longitudinal tube with an inner (cylindrical) layer which is impermeable to air and an outer (cylindrical) layer which is permeable to airflow, wherein there are holes in the inner layer to disperse air out along the length of the tube. In an example, the inner and outer layers can be nested and/or concentric. In an example, an intra-bed flow channel can be longitudinally tapered.

In an example, a bedding component can have longitudinal air-permeable channels along portions of its perimeter, wherein one or more intra-bed flow channels can be removably inserted (e.g. slid) into these channels. In an example, one or more air-conducting tubes can be removably inserted into longitudinal channels (e.g. fabric channels) along the side and foot sections of the perimeter of a bedding component. In an alternative example, a device or system to change, manage, and/or control the climate of one or more areas (e.g. zones) of a bed can comprise intra-bed flow channels, but not intra-room flow channels. In an example, such a device or system can exhaust air from intra-bed flow channels into ambient air in the bedroom instead of into to a central HVAC vent. In an example, such a device or system can extract heat from liquid circulated through intra-bed flow channels and transfer that heat into ambient air in a bedroom.

In an example, there can be multiple valves around (a portion of) the perimeter of a bed or bedding component. For example, if a person on one side of a bed prefers to have more airflow near their lower body and a person on the other side of the bed prefers to have more airflow near their upper body, then they can adjust these valves to create this airflow pattern. In an example, one valve can control flow through multiple holes or flow loops in an intra-bed flow channel. In an example, valves can be manually opened or closed. In another example, valves can be automatically opened or closed, wherein this automatic adjustment is controlled by a data processor and/or wireless remote control.

In an example, a device or system can include a plurality of valves which change flow rates of the flowable substance to different areas (e.g. sides or zones) of a bed. This allows a person to customize which portions of their body receive more or less airflow, based on their own preference. In an example, there can be between 3 and 6 valves on each lateral (e.g. right and left) side of a bed or bedding component, wherein these valves adjust the flow of a flowable substance through the bedding component and/or into a space in the bed. In an example, there can be a plurality of on each lateral (e.g. right and left) side of a bed or bedding component, wherein these valves adjust the flow of air from intra-bed flow channels into a space in the bed. In an example, there can be a plurality of on each lateral (e.g. right and left) side of a bed or bedding component, wherein these valves adjust the flows of a liquid (e.g. water) through intra-bed flow channels into different areas (e.g. sides or zones) of the bed.

In an example, a device or system can further comprise a flow-accelerating component (e.g. a blower, fan, turbine, or impeller). In an example, a device or system can further comprise a flow-accelerating component (e.g. a blower, fan, turbine, or impeller) which is near (e.g. within two feet of) a central HVAC vent. In an example, a flow-accelerating component can be a blower, fan, turbine, impellor, or pump. In an example, there can be a flow-accelerating component within an intra-room flow channel. In an example, there can be a flow-accelerating component near (e.g. within one foot of) a bed. In an example, there can be a flow-accelerating component in a housing along the side of the bed. In an example, there can be a flow-accelerating component in a housing in a bed mattress.

In an example, a device or system can comprise two or more flow-accelerating components which accelerate flows of a flowable substance through two or more intra-bed flow channels, respectively. In an example, flow rates through different fluid flow loops (e.g. flow circuits) through intra-bed flow channels in a bedding component can be independently-adjusted by adjusting different flow-accelerating components. In an example, a cooling component can cool a flowable substance (e.g. air or water) before it is conducted through an intra-bed flow channel. In an example, a cooling component can cool a flowable substance by extracting heat from a substance and then transferring that heat to air outside a bed. In an example, a cooling component can cool a flowable substance by extracting heat from a substance and then transferring that heat to a liquid outside a bed. In an example, a near-vent component can be a heat exchange unit. In an example, a heat exchange unit can be near (e.g. within two feet of) a bed.

In an example, a heating component can heat a flowable substance (e.g. air or water) before it is conducted through an intra-bed flow channel. In an example, a heating component can comprise a heat exchange unit. In an example, a heating component can heat a flowable substance by extracting heat from a liquid outside a bed and then transferring that heat to a flowable substance before it is conducted through an intra-bed flow channel. In an example, there can be a cooling component within an intra-room flow channel. In an example, there can be a cooling component near (e.g. within one foot of) a bed. In an example, there can be a cooling component in a housing along the side of the bed. In an example, there can be a cooling component in a housing in a bed mattress.

In an example, there can be a heating component within an intra-room flow channel. In an example, there can be a heating component near (e.g. within one foot of) a bed. In an example, there can be a heating component in a housing along the side of the bed. In an example, there can be a heating component in a housing in a bed mattress. In an example, a device or system can comprise two or more cooling components which decrease the temperatures of a flowable substance which flows through two or more intra-bed flow channels, respectively. In an example, temperatures of a flowable substance in different fluid flow loops (e.g. flow circuits) through intra-bed flow channels in a bedding component can be independently-adjusted by adjusting different cooling components.

In an example, a device or system can comprise two or more heating components which increase the temperatures of a flowable substance which flows through two or more intra-bed flow channels, respectively. In an example, temperatures of a flowable substance in different fluid flow loops (e.g. flow circuits) through intra-bed flow channels in a bedding component can be independently-adjusted by adjusting different heating components. In an example, a device or system can further comprise one or more moisture and/or humidity sensors, wherein the system stops the flow of a liquid flowable substance through a selected fluid flow loop or circuit if the sensors detect abnormally high moisture and/or humidity levels concerning (e.g. near) that loop or circuit.

In an example, a leak mitigation mechanism can comprise stopping the flow of a flowable substance through an intra-bed flow channel if an abnormal (e.g. high) amount of moisture and/or humidity is detected near the intra-bed flow channels. In an example, a leak mitigation mechanism can comprise stopping the flow of a flowable substance through an intra-bed flow channel if an abnormal (e.g. high) amount of moisture and/or humidity is detected in the intra-room flow channels. In an example, a leak mitigation mechanism can comprise stopping the flow of a flowable substance through an intra-bed flow channel if an abnormal (e.g. low) pressure of the liquid flowable substance is detected.

In an example, a device or system can further comprise one or more moisture and/or humidity sensors, wherein the system stops the flow of a liquid flowable substance through a selected fluid flow loop or circuit if the sensors detect abnormally rapid increases in moisture and/or humidity concerning (e.g. near) that loop or circuit. In an example, a leak mitigation mechanism can comprise stopping the flow of a flowable substance through an intra-bed flow channel if an abnormal (e.g. rapid) increase in moisture and/or humidity is detected near the intra-bed flow channels. In an example, a leak mitigation mechanism can comprise stopping the flow of a flowable substance through an intra-bed flow channel if an abnormal (e.g. rapid) increase in moisture and/or humidity is detected in the intra-room flow channels. In an example, a leak mitigation mechanism can comprise stopping the flow of a flowable substance through an intra-bed flow channel if an abnormal (e.g. rapid) decrease in the pressure of the liquid flowable substance is detected.

In an example, a device or system can further comprise one or more moisture and/or humidity sensors, wherein the system suctions a liquid flowable substance out from a selected fluid flow loop or circuit if the sensors detect abnormally high moisture and/or humidity levels concerning (e.g. near) that loop or circuit. In an example, a leak mitigation mechanism can comprise suctioning a flowable substance out from an intra-bed flow channel if an abnormal (e.g. high) amount of moisture and/or humidity is detected near the intra-bed flow channels. In an example, a leak mitigation mechanism can comprise suctioning a flowable substance out from an intra-bed flow channel if an abnormal (e.g. high) amount of moisture and/or humidity is detected in the intra-room flow channels. In an example, a mattress leak can really suck so a leak mitigation mechanism really sucks to avoid mattress leaks.

In an example, a device or system which changes, manages, and/or controls the climate of a bed using a liquid flowable substance can further comprise a leak mitigation mechanism. In an example, a device or system can further comprise one or more moisture and/or humidity sensors, wherein the system suctions a liquid flowable substance out from a selected fluid flow loop or circuit if the sensors detect abnormally rapid decreases in fluid pressure or quantity concerning (e.g. near) that loop or circuit. In an example, a leak mitigation mechanism can comprise suctioning a flowable substance out from an intra-bed flow channel if an abnormal (e.g. rapid) increase in moisture and/or humidity is detected in a bed. In an example, a leak mitigation mechanism can comprise suctioning a flowable substance out from an intra-bed flow channel if an abnormal (e.g. rapid) decrease in the quantity of the liquid flowable substance is detected.

In an example, a device or system to change, manage, and/or control the climate of one or more areas (e.g. zones) of a bed using a flow of liquid through intra-bed flow channels can have a leak mitigation mechanism comprising a plurality of liquid flow compartments which can be selectively sealed off in the event of a leak. In an example, a leak in a particular section can be detected by a means selected from the group consisting of: (rapid) decrease in liquid pressure, (rapid) decrease in liquid quantity, and (rapid) increase in moisture or humidity near (e.g. under or around) the section. In an example, this device or system can further comprise a dehumidifying component. In an example, this device or system can further comprise a local dehumidifying component which is located in an intra-room flow channel. In an example, this device or system can further comprise a dehumidifying component which extracts water from air that has been drawn out from a bed (e.g. out from the space between bottom and upper sheets).

In an example, this device or system can further comprise a flowable substance reservoir which contains an amount of the flowable substance. In an example, the amount of flowable substance in this reservoir can be monitored and the device or system can send an alert if this amount decreases rapidly (e.g. a more than a selected rate) which may indicate leakage. In an example, a device or system which changes, manages, and/or controls the climate of one or more areas (e.g. zones) of a bed using a liquid flowable substance can further comprise a disinfectant reservoir which contains an amount of disinfecting (e.g. anti-microbial) substance. In an example, a device or system can have sensors which are in fluid communication with a liquid flowable substance, wherein disinfectant is automatically dispensed into the liquid flowable substance when microbial growth is detected in the substance.

In an example, a wireless remote control device can be a held-held device. In an example, a wireless remote control device can be a (soft) hand-held device with a function (e.g. control of heating or cooling or airflow rate) which is activated by compression. In an example, a wireless remote control device can be a (soft) hand-held device with a function (e.g. control of heating or cooling or airflow rate) which is activated by compression, wherein a person in bed can change the rate of airflow through the bed by compressing (or releasing) the device. In an example, a wireless remote control device can be a (soft) hand-held device with a function (e.g. control of heating or cooling or airflow rate) which is activated by compression, wherein a person in bed can change the flow rate of a flowable substance through the bed by compressing (or releasing) the device.

In an example, data from sensors on the wearable item can be used to help determine what a person's phase and/or stage of sleep. In an example, a device or system which changes, manages, and/or controls the climate of one or more areas (e.g. zones) of a bed can further comprise a data processor, data processing unit, and/or central processing unit. In an example, this device or system can further comprise a wireless data receiver. In an example, data from these one or more sensors can be used to manage and/or control the climate of one or more areas (e.g. zones) of the bed as part of a closed (or partially-closed) feedback loop system. This concludes the introductory section and begins individual narratives for FIGS. 1 through 38.

FIGS. 1 and 2 show two views, from two different perspectives, of an example of a device or system which changes, manages, and/or controls the climate (e.g. temperature and/or humidity) of one or more areas (e.g. sides or zones) of a bed using a flowable substance. In this example, the flowable substance is air. In this example, an intra-room flow channel (between a bed and an airflow vent) is substantially co-planar with (e.g. flat against) bedroom walls. FIG. 1 shows this example from an oblique side perspective, including a bed along one wall and vent on another wall. FIG. 2 shows this example from a top-down cross-sectional perspective, including more detail concerning intra-bed flow channels.

The device or system to change, manage, and/or control the climate of one or more areas (e.g. sides or zones) of a bed shown in FIGS. 1 and 2 comprises: one or more intra-room flow channels 102 which conduct a flowable substance between a first component and a second component in a bedroom (e.g. room containing a bed), wherein the first component is (a) an airflow vent 103 (e.g. on a room floor, wall, or ceiling) though which air enters the room from a central HVAC system, (b) an airflow vent (e.g. on a room floor, wall, or ceiling) through which air exits the room to a central HVAC system, or (c) a window-mounted air conditioner, and wherein the second component is a bedding component 101 (e.g. a bed mattress, mattress pad or topper, lower and/or fitted bed sheet, upper bed sheet, blanket, comforter, quilt, or other specialized bedding); and one or more intra-bed flow channels (including channel 201) which conduct the flowable substance through the bedding component in order to change the climate (e.g. temperature and/or humidity) of one or more areas (e.g. sides or zones) of the bed. This example also includes a plurality of valves (including valve 202) which change flow rates of the flowable substance to different areas (e.g. zones), respectively, of the bed. Example variations discussed elsewhere in this disclosure or priority-linked disclosures can also be applied to this example where relevant.

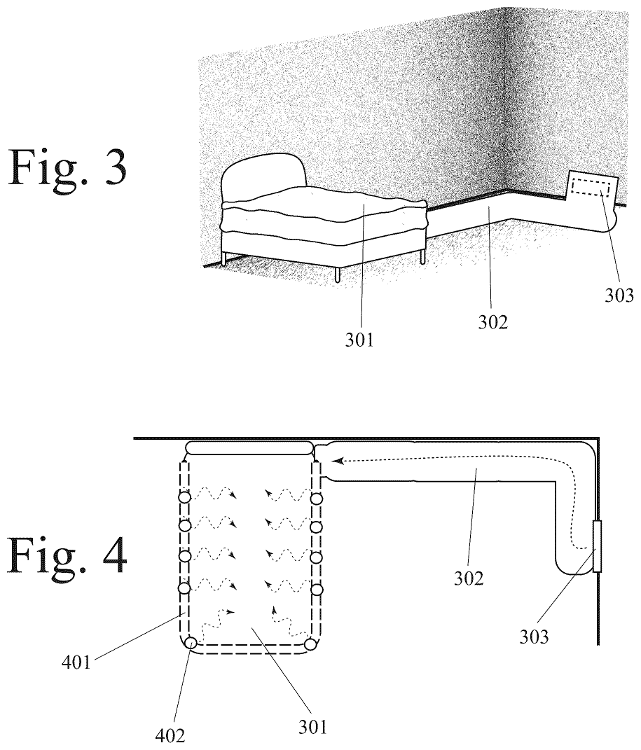

FIGS. 3 and 4 show two views, from two different perspectives, of an example of a device or system which changes, manages, and/or controls the climate (e.g. temperature and/or humidity) of one or more areas (e.g. sides or zones) of a bed using a flowable substance. In this example, the flowable substance is air. In this example, an intra-room flow channel (between a bed and an airflow vent) is substantially co-planar with (e.g. flat against) a bedroom floor, except for a vertical portion over a wall vent. FIG. 3 shows this example from an oblique side perspective, including a bed along one wall and vent on another wall. FIG. 4 shows this example from a top-down cross-sectional perspective, including more detail concerning intra-bed flow channels.

The device or system to change, manage, and/or control the climate of one or more areas (e.g. sides or zones) of a bed shown in FIGS. 3 and 4 comprises: one or more intra-room flow channels 302 which conduct a flowable substance between a first component and a second component in a bedroom (e.g. room containing a bed), wherein the first component is (a) an airflow vent 303 (e.g. on a room floor, wall, or ceiling) though which air enters the room from a central HVAC system, (b) an airflow vent (e.g. on a room floor, wall, or ceiling) through which air exits the room to a central HVAC system, or (c) a window-mounted air conditioner, and wherein the second component is a bedding component 301 (e.g. a bed mattress, mattress pad or topper, lower and/or fitted bed sheet, upper bed sheet, blanket, comforter, quilt, or other specialized bedding); and one or more intra-bed flow channels (including channel 401) which conduct the flowable substance through the bedding component in order to change the climate (e.g. temperature and/or humidity) of one or more areas (e.g. sides or zones) of the bed. This example also includes a plurality of valves (including valve 402) which change flow rates of the flowable substance to different areas (e.g. zones), respectively, of the bed. Example variations discussed elsewhere in this disclosure or priority-linked disclosures can also be applied to this example where relevant.

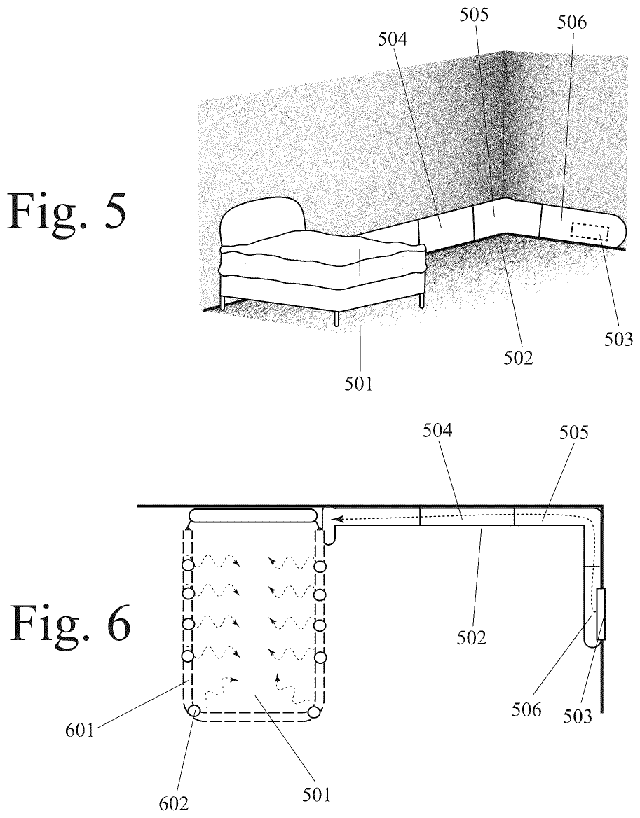

FIGS. 5 and 6 show two views, from two different perspectives, of an example of a device or system which changes, manages, and/or controls the climate (e.g. temperature and/or humidity) of one or more areas (e.g. sides or zones) of a bed using a flowable substance. In this example, the flowable substance is air. In this example, an intra-room flow channel (between a bed and an airflow vent) is comprised of connected modular sections. FIG. 5 shows this example from an oblique side perspective, including a bed along one wall and vent on another wall. FIG. 6 shows this example from a top-down cross-sectional perspective, including more detail concerning intra-bed flow channels.

The device or system to change, manage, and/or control the climate of one or more areas (e.g. sides or zones) of a bed shown in FIGS. 5 and 6 comprises: one or more intra-room flow channels 502 which conduct a flowable substance between a first component and a second component in a bedroom (e.g. room containing a bed); wherein the first component is (a) an airflow vent 503 (e.g. on a room floor, wall, or ceiling) though which air enters the room from a central HVAC system, (b) an airflow vent (e.g. on a room floor, wall, or ceiling) through which air exits the room to a central HVAC system, or (c) a window-mounted air conditioner; wherein the second component is a bedding component 501 (e.g. a bed mattress, mattress pad or topper, lower and/or fitted bed sheet, upper bed sheet, blanket, comforter, quilt, or other specialized bedding); and wherein the one or more intra-room flow channels are comprised of one or more straight modular sections (including 504) along a wall, one or more corner modular sections (505) around a room corner, and/or one or vent modular sections (506) which go on a vent; and one or more intra-bed flow channels (including channel 601) which conduct the flowable substance through the bedding component in order to change the climate (e.g. temperature and/or humidity) of one or more areas (e.g. sides or zones) of the bed. This example also includes a plurality of valves (including valve 602) which change flow rates of the flowable substance to different areas (e.g. zones), respectively, of the bed. Example variations discussed elsewhere in this disclosure or priority-linked disclosures can also be applied to this example where relevant.

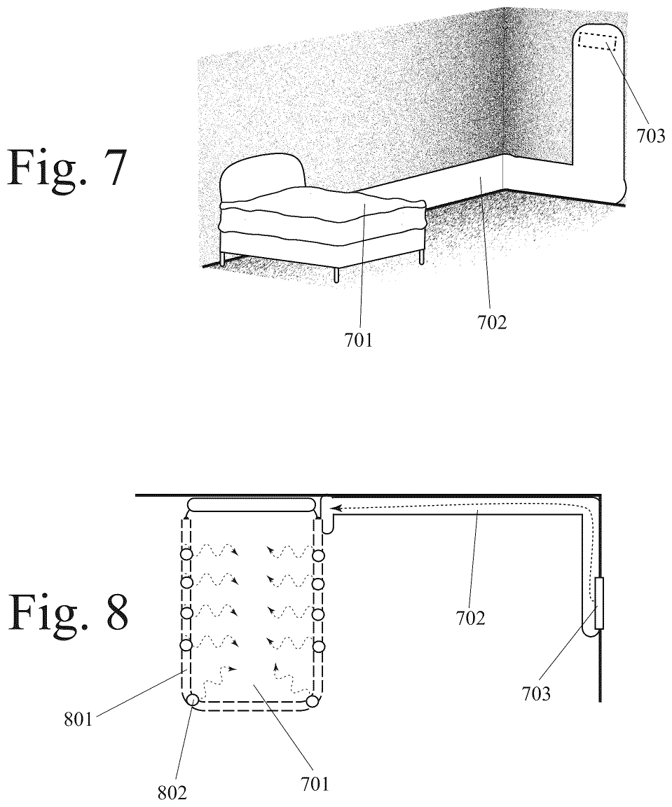

FIGS. 7 and 8 show two views, from two different perspectives, of an example of a device or system which changes, manages, and/or controls the climate (e.g. temperature and/or humidity) of one or more areas (e.g. sides or zones) of a bed using a flowable substance. In this example, the flowable substance is air. In this example, an intra-room flow channel (between a bed and an airflow vent) is substantially co-planar with (e.g. flat against) bedroom walls. In this example, the vent is on an upper portion of a wall. FIG. 7 shows this example from an oblique side perspective, including a bed along one wall and vent on another wall. FIG. 8 shows this example from a top-down cross-sectional perspective, including more detail concerning intra-bed flow channels.

The device or system to change, manage, and/or control the climate of one or more areas (e.g. sides or zones) of a bed shown in FIGS. 7 and 8 comprises: one or more intra-room flow channels 702 which conduct a flowable substance between a first component and a second component in a bedroom (e.g. room containing a bed), wherein the first component is (a) an airflow vent 703 (e.g. on a room floor, wall, or ceiling) though which air enters the room from a central HVAC system, (b) an airflow vent (e.g. on a room floor, wall, or ceiling) through which air exits the room to a central HVAC system, or (c) a window-mounted air conditioner, and wherein the second component is a bedding component 701 (e.g. a bed mattress, mattress pad or topper, lower and/or fitted bed sheet, upper bed sheet, blanket, comforter, quilt, or other specialized bedding); and one or more intra-bed flow channels (including channel 801) which conduct the flowable substance through the bedding component in order to change the climate (e.g. temperature and/or humidity) of one or more areas (e.g. sides or zones) of the bed. This example also includes a plurality of valves (including valve 802) which change flow rates of the flowable substance to different areas (e.g. zones), respectively, of the bed. Example variations discussed elsewhere in this disclosure or priority-linked disclosures can also be applied to this example where relevant.

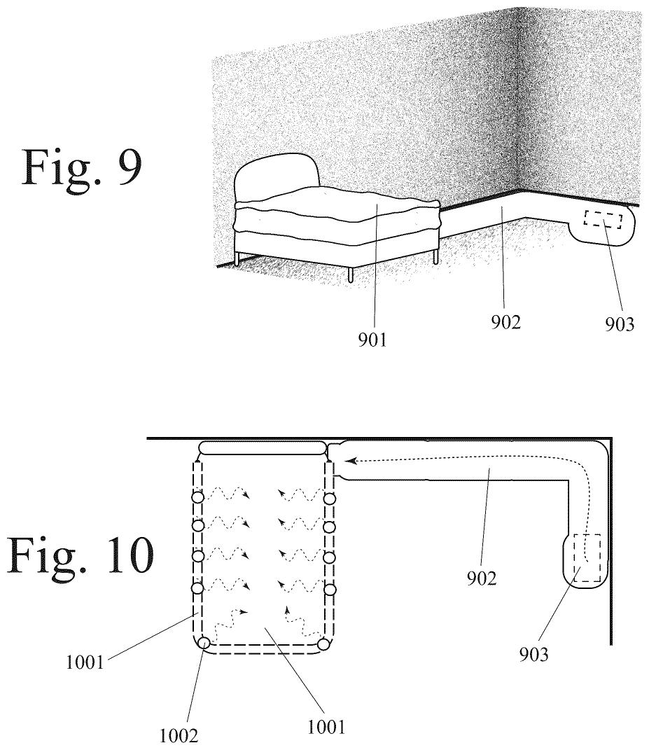

FIGS. 9 and 10 show two views, from two different perspectives, of an example of a device or system which changes, manages, and/or controls the climate (e.g. temperature and/or humidity) of one or more areas (e.g. sides or zones) of a bed using a flowable substance. In this example, the flowable substance is air. In this example, an intra-room flow channel (between a bed and an airflow vent) is substantially co-planar with (e.g. flat against) a bedroom floor. In this example, the vent is on the bedroom floor. FIG. 9 shows this example from an oblique side perspective, including a bed along one wall and a vent on the floor. FIG. 10 shows this example from a top-down cross-sectional perspective, including more detail concerning intra-bed flow channels.

The device or system to change, manage, and/or control the climate of one or more areas (e.g. sides or zones) of a bed shown in FIGS. 9 and 10 comprises: one or more intra-room flow channels 902 which conduct a flowable substance between a first component and a second component in a bedroom (e.g. room containing a bed), wherein the first component is (a) an airflow vent 903 (e.g. on a room floor, wall, or ceiling) though which air enters the room from a central HVAC system, (b) an airflow vent (e.g. on a room floor, wall, or ceiling) through which air exits the room to a central HVAC system, or (c) a window-mounted air conditioner, and wherein the second component is a bedding component 901 (e.g. a bed mattress, mattress pad or topper, lower and/or fitted bed sheet, upper bed sheet, blanket, comforter, quilt, or other specialized bedding); and one or more intra-bed flow channels (including channel 1001) which conduct the flowable substance through the bedding component in order to change the climate (e.g. temperature and/or humidity) of one or more areas (e.g. sides or zones) of the bed. This example also includes a plurality of valves (including valve 1002) which change flow rates of the flowable substance to different areas (e.g. zones), respectively, of the bed. Example variations discussed elsewhere in this disclosure or priority-linked disclosures can also be applied to this example where relevant.

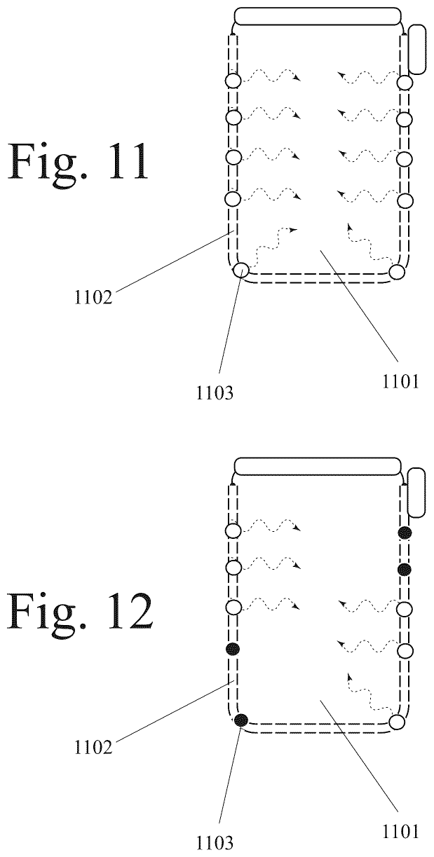

FIGS. 11 and 12 show two views, at two different times, of an example of a device or system which changes, manages, and/or controls the climate (e.g. temperature and/or humidity) of one or more areas (e.g. sides or zones) of a bed using a flowable substance. In this example, the flowable substance is air. In this example, different valves around the perimeter of a bed can be individually-adjusted to change the amounts of airflow to different areas (e.g. sides or zones) of a bed. This allows a person to customize which portions of their body receive more or less airflow, based on their own preference. It also allows two people on different sides of a bed to receive more or less airflow on different portions of their bodies, according to their different preferences. FIG. 11 shows this example at a first time when there are substantially even amounts of airflow to all portions of a bed. FIG. 12 shows this example at a second time when there is more airflow to upper body portions (of a first person) on the left side of the bed and more airflow to a lower body portions (of a second person) on the right side of the bed.

The device or system to change, manage, and/or control the climate of one or more areas (e.g. sides or zones) of a bed shown in FIGS. 11 and 12 comprises: one or more intra-bed flow channels (including channel 1102) which conduct a flowable substance through a bedding component 1101 (e.g. a bed mattress, mattress pad or topper, lower and/or fitted bed sheet, upper bed sheet, blanket, comforter, quilt, or other specialized bedding) in order to change the climate (e.g. temperature and/or humidity) of one or more areas (e.g. sides or zones) of the bed; and a plurality of individually-adjustable valves (including valve 1103) which change flow rates of the flowable substance to different areas (e.g. zones), respectively, of the bed. Example variations discussed elsewhere in this disclosure or priority-linked disclosures can also be applied to this example where relevant.

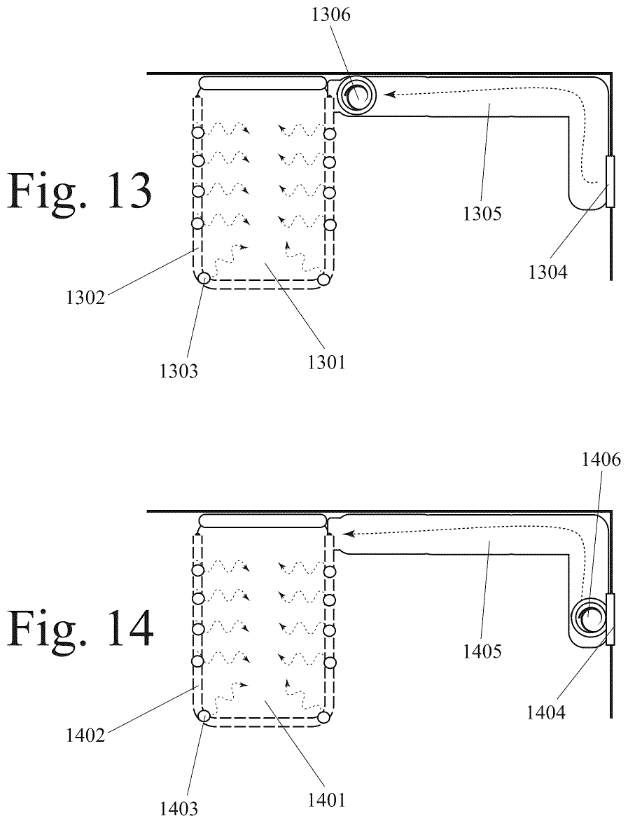

FIG. 13 shows a top-down view of an example of a device or system which changes, manages, and/or controls the climate (e.g. temperature and/or humidity) of one or more areas (e.g. sides or zones) of a bed using a flowable substance. In this example, the flowable substance is air. In this example, the device or system further comprises a flow-accelerating component (e.g. a blower, fan, turbine, or impeller) near the bed.

The device or system to change, manage, and/or control the climate of one or more areas (e.g. sides or zones) of a bed shown in FIG. 13 comprises: one or more intra-room flow channels 1305 which conduct a flowable substance between a first component and a second component in a bedroom (e.g. room containing a bed), wherein the first component is (a) an airflow vent 1304 (e.g. on a room floor, wall, or ceiling) though which air enters the room from a central HVAC system, (b) an airflow vent (e.g. on a room floor, wall, or ceiling) through which air exits the room to a central HVAC system, or (c) a window-mounted air conditioner, and wherein the second component is a bedding component 1301 (e.g. a bed mattress, mattress pad or topper, lower and/or fitted bed sheet, upper bed sheet, blanket, comforter, quilt, or other specialized bedding); a flow-accelerating component 1306 (e.g. a blower, fan, turbine, or impeller) near the second component; and one or more intra-bed flow channels (including channel 1302) which conduct the flowable substance through the bedding component in order to change the climate (e.g. temperature and/or humidity) of one or more areas (e.g. sides or zones) of the bed. This example also includes a plurality of valves (including valve 1303) which change flow rates of the flowable substance to different areas (e.g. zones), respectively, of the bed. Example variations discussed elsewhere in this disclosure or priority-linked disclosures can also be applied to this example where relevant.

FIG. 14 shows a top-down view of an example of a device or system which changes, manages, and/or controls the climate (e.g. temperature and/or humidity) of one or more areas (e.g. sides or zones) of a bed using a flowable substance. In this example, the device or system further comprises a flow-accelerating component (e.g. a blower, fan, turbine, or impeller) near a central HVAC vent.

The device or system to change, manage, and/or control the climate of one or more areas (e.g. sides or zones) of a bed shown in FIG. 14 comprises: one or more intra-room flow channels 1405 which conduct a flowable substance between a first component and a second component in a bedroom (e.g. room containing a bed), wherein the first component is (a) an airflow vent 1404 (e.g. on a room floor, wall, or ceiling) though which air enters the room from a central HVAC system, (b) an airflow vent (e.g. on a room floor, wall, or ceiling) through which air exits the room to a central HVAC system, or (c) a window-mounted air conditioner, and wherein the second component is a bedding component 1401 (e.g. a bed mattress, mattress pad or topper, lower and/or fitted bed sheet, upper bed sheet, blanket, comforter, quilt, or other specialized bedding); a flow-accelerating component 1406 (e.g. a blower, fan, turbine, or impeller) near the first component; and one or more intra-bed flow channels (including channel 1402) which conduct the flowable substance through the bedding component in order to change the climate (e.g. temperature and/or humidity) of one or more areas (e.g. sides or zones) of the bed. This example also includes a plurality of valves (including valve 1403) which change flow rates of the flowable substance to different areas (e.g. zones), respectively, of the bed. Example variations discussed elsewhere in this disclosure or priority-linked disclosures can also be applied to this example where relevant.

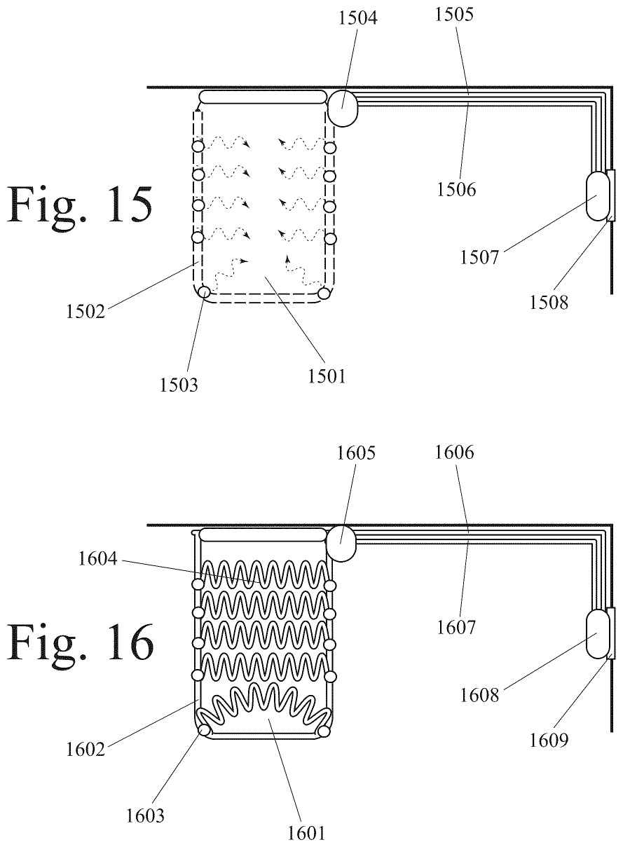

FIG. 15 shows a top down view of an example of a device or system which changes, manages, and/or controls the climate (e.g. temperature and/or humidity) of one or more areas (e.g. sides or zones) of a bed using both air and a liquid (e.g. water). In this example, the flowable substance in flow channels in a bedding component is air and the flowable substance in flow channels between the bed and a central HVAC vent is a liquid (e.g. water). This example also includes: a flow-accelerating component (e.g. a blower, fan, turbine, or impeller); and a heat exchange unit which transfers heat from the air to the liquid, or vice versa.

The device or system to change, manage, and/or control the climate of one or more areas (e.g. sides or zones) of a bed shown in FIG. 15 comprises: two intra-room flow channels (1505 and 1506) which conduct flows of a liquid (e.g. water) in two directions, respectively, between a near-vent component (e.g. within two feet of a central HVAC vent 1508) and a near-bed component (e.g. within two feet of a bed), wherein a first component selected from the near-vent component and the near-bed component is a heat exchange unit 1504 which transfers heat from the liquid to air (or vice versa), and wherein a second component selected from the near-vent component and the near-bed component is a flow-accelerating component 1507 (e.g. a blower, fan, turbine, impeller, or pump); and one or more intra-bed flow channels (including channel 1502) which conduct air flows through a bedding component 1501 (e.g. a bed mattress, mattress pad or topper, lower and/or fitted bed sheet, upper bed sheet, blanket, comforter, quilt, or other specialized bedding), wherein the air flows are heated or cooled by the heat exchange unit in order to change the climate (e.g. temperature and/or humidity) of one or more areas (e.g. sides or zones) of the bed. This example also includes a plurality of valves (including valve 1503) which change flow rates of air to different areas (e.g. zones), respectively, of the bed. Example variations discussed elsewhere in this disclosure or priority-linked disclosures can also be applied to this example where relevant.

FIG. 16 shows a top down view of an example of a device or system which changes, manages, and/or controls the climate (e.g. temperature and/or humidity) of one or more areas (e.g. sides or zones) of a bed using a liquid (e.g. water) in both intra-room flow channels and intra-bed flow channels. This example includes: a flow-accelerating component (e.g. a blower, fan, turbine, impeller, or pump); and a heat exchange unit which transfers heat from air to liquid, or vice versa.

The device or system to change, manage, and/or control the climate of one or more areas (e.g. sides or zones) of a bed shown in FIG. 16 comprises: two intra-room flow channels (1606 and 1607) which conduct flows of a liquid (e.g. water) in two directions, respectively, between a heat exchange unit 1608 and a flow-accelerating component (e.g. a blower, fan, turbine, impeller, or pump) 1605; and one or more intra-bed flow channels (including channels 1602 and 1604) which conduct flows of liquid through a bedding component 1601 (e.g. a bed mattress, mattress pad or topper, lower and/or fitted bed sheet, upper bed sheet, blanket, comforter, quilt, or other specialized bedding) in order to change the climate (e.g. temperature and/or humidity) of one or more areas (e.g. sides or zones) of the bed. This example also includes a plurality of valves (including valve 1603) which change flow rates of the liquid through different areas (e.g. zones), respectively, of the bed. FIG. 16 also shows central HVAC vent 1609. Example variations discussed elsewhere in this disclosure or priority-linked disclosures can also be applied to this example where relevant.

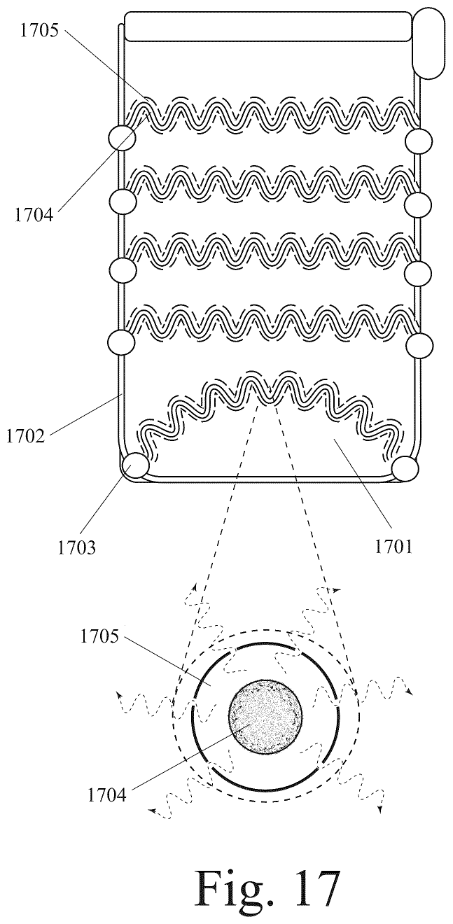

FIG. 17 shows an example of a hybrid device or system which changes, manages, and/or controls the climate (e.g. temperature and/or humidity) of one or more areas (e.g. sides or zones) of a bed using both a liquid (e.g. water) and air. In this example, a liquid flow channel is within an air flow channel. This hybrid liquid-air design may be able to combine the best features of liquid flow and air flow for managing bed climate. For example, this hybrid design may be able to combine the thermal-management efficiency of liquid flow and the moisture-removing benefits of air flow. It can also help to mitigate possible leakage of liquid as compared to liquid-only designs. The upper portion of FIG. 17 shows a top-down view of a bed (portion of) this hybrid device or system. The lower portion of FIG. 17 shows an expanded cross-sectional view of an intra-bed flow channel in this hybrid device or system.

The hybrid device or system to change, manage, and/or control the climate of one or more areas (e.g. sides or zones) of a bed shown in FIG. 17 comprises: one or more intra-bed flow channels (including channel 1702) which conduct flows of both liquid and air through a bedding component 1701 (e.g. a bed mattress, mattress pad or topper, lower and/or fitted bed sheet, upper bed sheet, blanket, comforter, quilt, or other specialized bedding) in order to change the climate (e.g. temperature and/or humidity) of one or more areas (e.g. sides or zones) of the bed, wherein an intra-bed flow channel further comprises both a liquid flow channel 1704 and an air flow channel 1705. In this example, the liquid flow channel is within the air flow channel and there are openings in the perimeter of the air flow channel to let air escape. This example also includes a plurality of valves (including valve 1703) which change flow rates of liquid and/or air to different areas (e.g. zones), respectively, of the bed.

In an example, a device or system can be a hybrid device or system which uses both air flows and liquid flows. In an example, intra-bed flow channels can conduct liquid through an interior flow channel and can conduct air through an outer flow channel, wherein the interior flow channel is nested within the outer flow channel. Such a hybrid (air flow and liquid flow) system can combine the temperature-control efficiency of liquid flow and the moisture-removing benefit of air flow. Example variations discussed elsewhere in this disclosure or priority-linked disclosures can also be applied to this example where relevant.

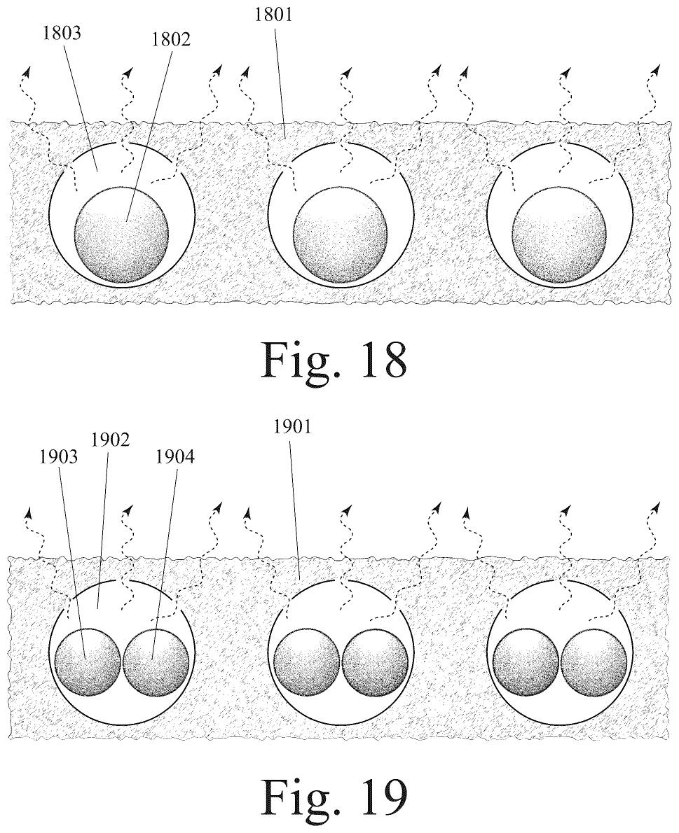

FIG. 18 shows a cross-sectional view of a hybrid climate-control bedding component (e.g. mattress or mattress pad) with a plurality of intra-bed flow channels which conduct flows of both liquid (e.g. water) and air. In this example, liquid-flow channels are nested within air-flow channels. In an example, cooled or heated liquid in the liquid-flow channels cools or heats air in the surrounding air-flow channels. This hybrid liquid-air design may be able to combine the best features of liquid flow and air flow for managing bed climate. For example, this hybrid design may be able to combine the thermal-management efficiency of liquid flow and the moisture-removing benefits of air flow. It can also help to mitigate possible leakage of liquid as compared to liquid-only designs.

The hybrid device or system to change, manage, and/or control the climate of one or more areas (e.g. sides or zones) of a bed shown in FIG. 18 comprises: a bedding component 1801 (e.g. a bed mattress, mattress pad or topper, lower and/or fitted bed sheet, upper bed sheet, blanket, comforter, quilt, or other specialized bedding); one or more intra-bed flow channels which conduct flows of both liquid and air through the bedding component, wherein an intra-bed flow channel further comprises both a liquid flow channel 1802 and an air flow channel 1803. In this example, the liquid flow channel is nested within the air flow channel. In this example, there are openings in the upper perimeter of the air flow channel which let air escape through the upper surface of the mattress.

In an example, a device or system can be a hybrid device or system which uses both air flows and liquid flows. In an example, intra-bed flow channels can conduct liquid through an interior flow channel and can conduct air through an outer flow channel, wherein the interior flow channel is nested within the outer flow channel. Such a hybrid (air flow and liquid flow) system can combine the temperature-control efficiency of liquid flow and the moisture-removing benefit of air flow. Example variations discussed elsewhere in this disclosure or priority-linked disclosures can also be applied to this example where relevant.

FIG. 19 shows a cross-sectional view of a hybrid climate-control bedding component (e.g. mattress or mattress pad) with a plurality of intra-bed flow channels which conduct flows of both liquid (e.g. water) and air. In this example, liquid-flow channels are nested within air-flow channels. In an example, cooled or heated liquid in the liquid-flow channels cools or heats air in the surrounding air-flow channels. This hybrid liquid-air design may be able to combine the best features of liquid flow and air flow for managing bed climate. For example, this hybrid design may be able to combine the thermal-management efficiency of liquid flow and the moisture-removing benefits of air flow. It can also help to mitigate possible leakage of liquid as compared to liquid-only designs.

The hybrid device or system to change, manage, and/or control the climate of one or more areas (e.g. sides or zones) of a bed shown in FIG. 19 comprises: a bedding component 1901 (e.g. a bed mattress, mattress pad or topper, lower and/or fitted bed sheet, upper bed sheet, blanket, comforter, quilt, or other specialized bedding); one or more intra-bed flow channels which conduct flows of both liquid and air through the bedding component, wherein an intra-bed flow channel further comprises multiple liquid flow channels 1903 and 1904 within an air flow channel 1902. In this example, there are openings in the upper perimeter of the air flow channel which let air escape through the upper surface of the mattress.

In an example, a device or system can be a hybrid device or system which uses both air flows and liquid flows. In an example, intra-bed flow channels can conduct liquid through an interior flow channel and can conduct air through an outer flow channel, wherein the interior flow channel is nested within the outer flow channel. Such a hybrid (air flow and liquid flow) system can combine the temperature-control efficiency of liquid flow and the moisture-removing benefit of air flow.

Example variations discussed elsewhere in this disclosure or priority-linked disclosures can also be applied to this example where relevant.

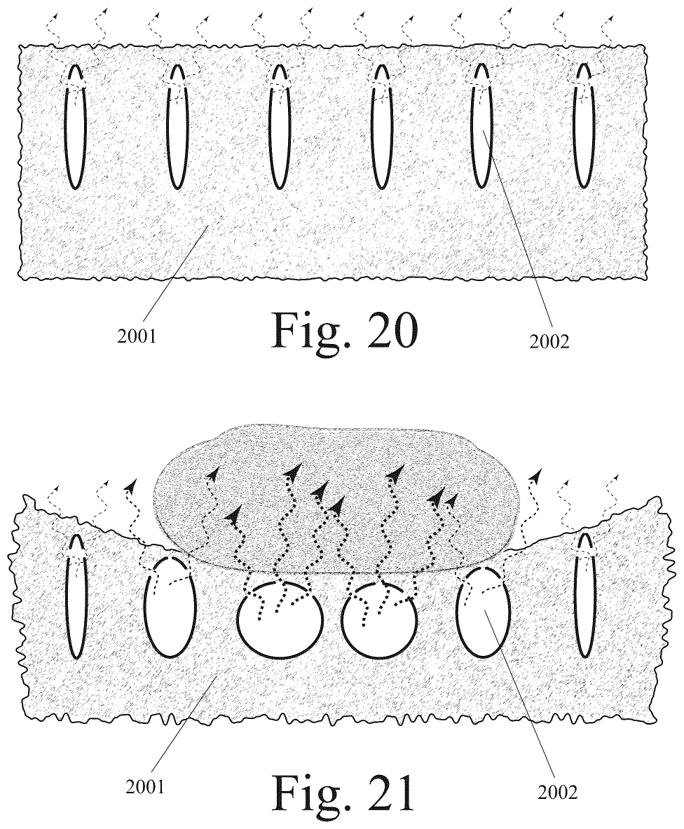

FIGS. 20 and 21 shows two cross-sectional views, at two different times, of a bedding component (e.g. mattress or mattress pad) with a plurality of intra-bed airflow channels, wherein channels which are directly under a sleep person's body release greater airflow than channels which are not directly under the sleeping person's body. This functionality can be especially useful for preventing the accumulation of moisture and/or warmth between a person's body and a mattress which can cause bed ulcers. It also can be more energy efficient for cooling or heating a person's body than designs which cool or heat all areas of a bed. FIG. 20 shows this example at a first time, before there is a person on top of the bedding component. FIG. 21 shows this example at a second time, when there is a person on top of the bedding component and when pressure from the person's body has selectively increased airflow from airflow channels which are directly below the person's body.

The device or system to change, manage, and/or control the climate of one or more areas (e.g. sides or zones) of a bed shown in FIGS. 20 and 21 comprises: a bedding component 2001 (e.g. a bed mattress, mattress pad or topper, lower and/or fitted bed sheet, upper bed sheet, blanket, comforter, quilt, or other specialized bedding); one or more intra-bed flow channels (including 2002) which conduct airflows through the bedding component, wherein the intra-bed flow channels are designed so flow channels which are directly below a person's body release more air than flow channels which are not directly below the person's body.

In this example, as shown in FIG. 20, intra-bed flow channels can have first configurations with narrow (e.g. elliptical) cross-sectional shapes in the absence of pressure from a person's body, wherein these flow channels conduct and release a first amount of airflow into a bed space (e.g. between a bottom sheet and a top sheet). In this example, as shown in FIG. 21, a subset of intra-bed flow channels which are directly under a person's body can have second configurations with wide (e.g. circular) cross-sectional shapes, wherein these flow channels conduct and release a second amount of airflow into a bed space (e.g. between a bottom sheet and a top sheet), and wherein the second amount is greater than the first amount. Example variations discussed elsewhere in this disclosure or priority-linked disclosures can also be applied to this example where relevant.

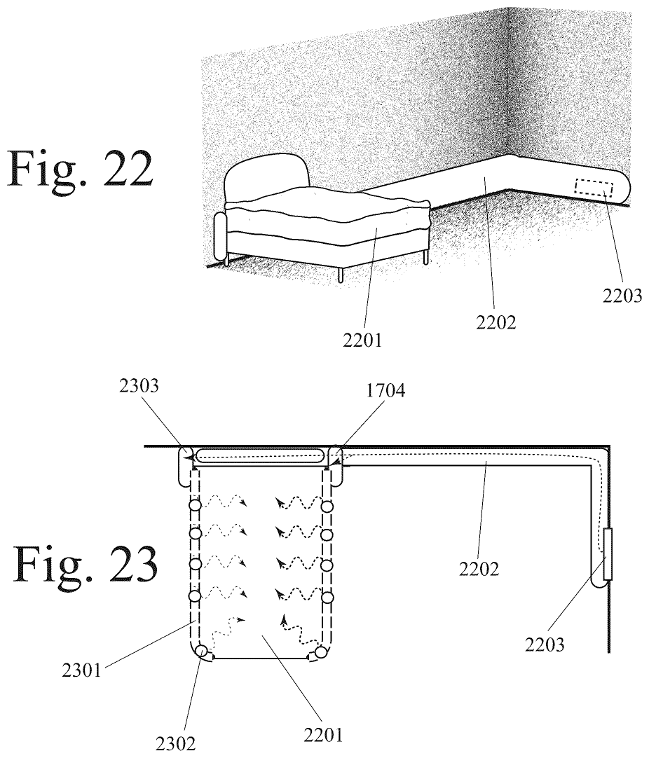

FIGS. 22 and 23 show two views, from two different perspectives, of an example of a device or system which changes, manages, and/or controls the climate (e.g. temperature and/or humidity) of one or more areas (e.g. sides or zones) of a bed using a flowable substance. In this example, the flowable substance is air. In this example, valves have been selectively adjusted to deliver greater airflow to the right side of the bed than the left side of the bed. FIG. 22 shows this example from an oblique side perspective. FIG. 23 shows this example from a top-down cross-sectional perspective.

The device or system to change, manage, and/or control the climate of one or more areas (e.g. sides or zones) of a bed shown in FIGS. 22 and 23 comprises: one or more intra-room flow channels 2202 which conduct a flowable substance between a first component and a second component in a bedroom (e.g. room containing a bed), wherein the first component is (a) an airflow vent 2203 (e.g. on a room floor, wall, or ceiling) though which air enters the room from a central HVAC system. (b) an airflow vent (e.g. on a room floor, wall, or ceiling) through which air exits the room to a central HVAC system, or (c) a window-mounted air conditioner, and wherein the second component is a bedding component 2201 (e.g. a bed mattress, mattress pad or topper, lower and/or fitted bed sheet, upper bed sheet, blanket, comforter, quilt, or other specialized bedding); one or more intra-bed flow channels (including channel 2301) which conduct the flowable substance through the bedding component in order to change the climate (e.g. temperature and/or humidity) of one or more areas (e.g. sides or zones) of the bed; and a plurality of valves (including valve 2302) which change flow rates of the flowable substance to different areas (e.g. zones), respectively, of the bed. Example variations discussed elsewhere in this disclosure or priority-linked disclosures can also be applied to this example where relevant.

FIG. 24 shows a cross-sectional view of an intra-room flow channel 2403 with an arcuate convex (e.g. circular, oval, or elliptical) cross-sectional shape. This figure also shows bedroom floor 2401 and bedroom wall 2402 to show the channel in context.

FIG. 25 shows a cross-sectional view of an intra-room flow channel 2503 with a quadrilateral (e.g. square, rectangular, or trapezoidal) cross-sectional shape. This figure also shows bedroom floor 2501 and bedroom wall 2502 to show the channel in context.

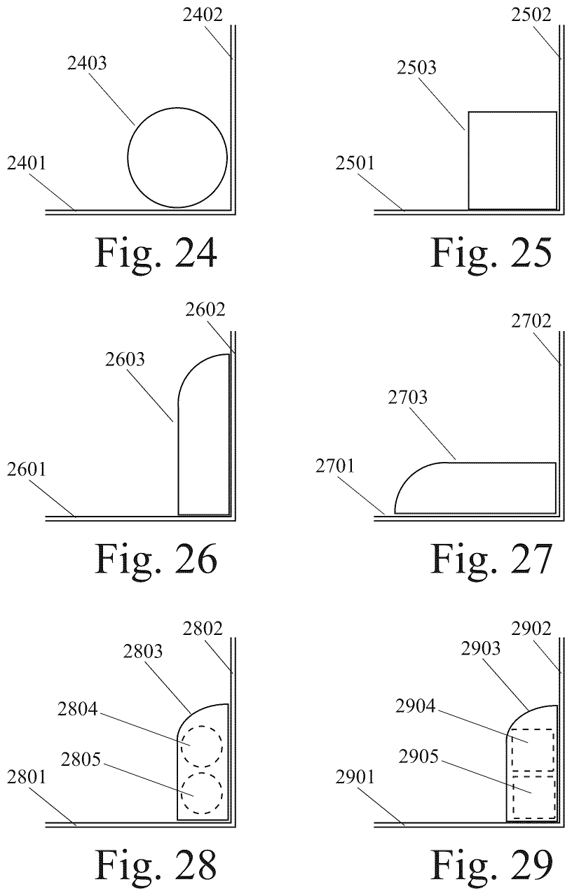

FIG. 26 shows a cross-sectional view of an intra-room flow channel 2603 with a rounded-quadrilateral (e.g. a quadrilateral with a rounded end or vertex) cross-sectional shape, wherein the rounded end or vertex is the upper side of the channel. This figure also shows bedroom floor 2601 and bedroom wall 2602 to show the channel in context.

FIG. 27 shows a cross-sectional view of an intra-room flow channel 2703 with a rounded-quadrilateral (e.g. a quadrilateral with a rounded end or vertex) cross-sectional shape, wherein the rounded end or vertex is a lateral side of the channel. This figure also shows bedroom floor 2701 and bedroom wall 2702 to show the channel in context.

FIG. 28 shows a cross-sectional view of housing 2803 which contains two intra-room flow channels (2804 and 2805) with arcuate cross-sectional shapes which conduct a flowable substance in two directions, respectively. This figure also shows bedroom floor 2801 and bedroom wall 2802 to show the channel in context.

FIG. 29 shows a cross-sectional view of housing 2903 which contains two intra-room flow channels (2904 and 2905) with polygonal cross-sectional shapes which conduct a flowable substance in two directions, respectively. This figure also shows bedroom floor 2901 and bedroom wall 2902 to show the channel in context.

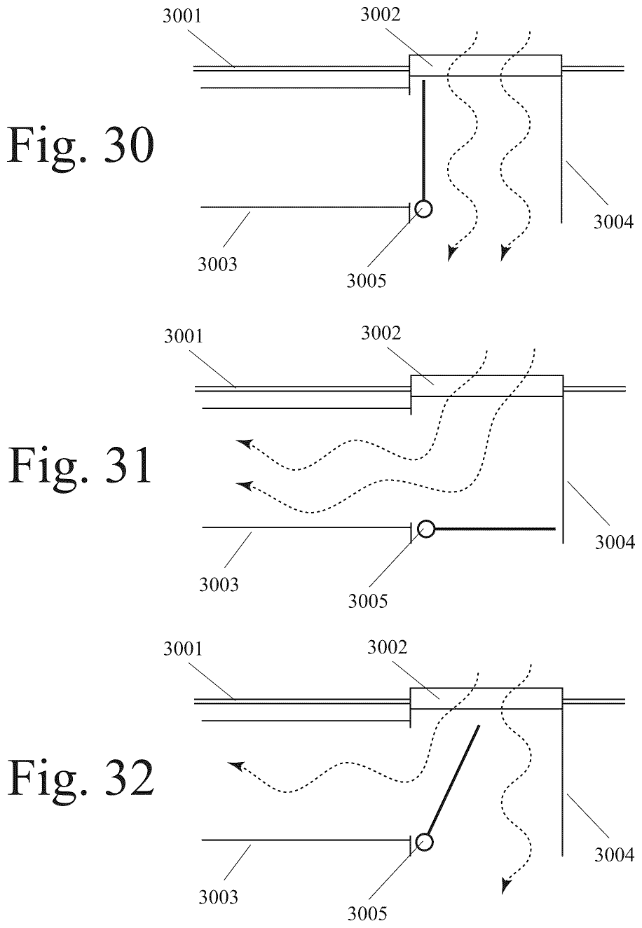

FIGS. 30 through 32 shows three top-down views, at three different times, of a section of an intra-room airflow channel which goes on a central HVAC vent, wherein this section includes a valve that adjusts how much air goes from the vent through an isolated channel to a bed (to change the climate of a space within the bed) versus how much air goes from the vent into ambient bedroom air. FIG. 30 shows this section at a first time, when all of the air coming out of the vent goes into ambient bedroom air. FIG. 31 shows this section at a second time, when all of the air coming out of the vent goes through an isolated channel to a bed. FIG. 32 shows this section at a third time, when some of the air from the vent goes through an isolated channel to a bed and some of the air from the vent goes into ambient bedroom air.

The section of an intra-room flow channel on a HVAC vent shown in FIGS. 30 through 32 comprises: a first flow channel branch 3004 which conducts air from the vent to ambient bedroom air; a second flow channel branch 3003 which conducts air from the vent to a bed; and a valve 3005 with a first configuration which opens the first flow channel branch and closes the second flow channel branch and a second configuration which closes the first flow channel branch and opens the second flow channel branch. This figure also shows room wall 3001 and HVAC vent 3002 for context. Example variations discussed elsewhere in this disclosure or priority-linked disclosures can also be applied to this example where relevant.

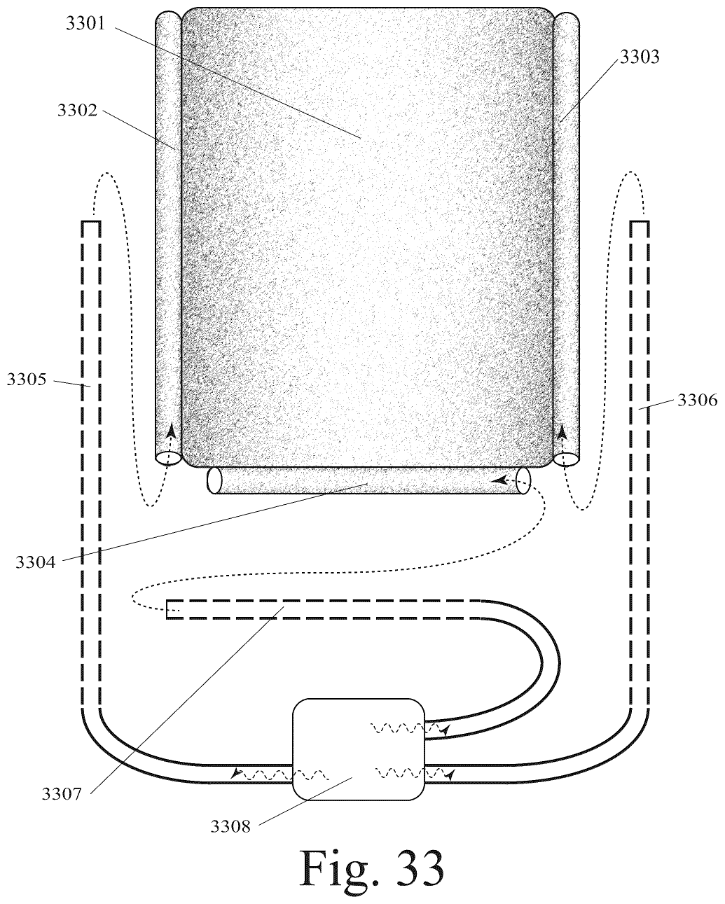

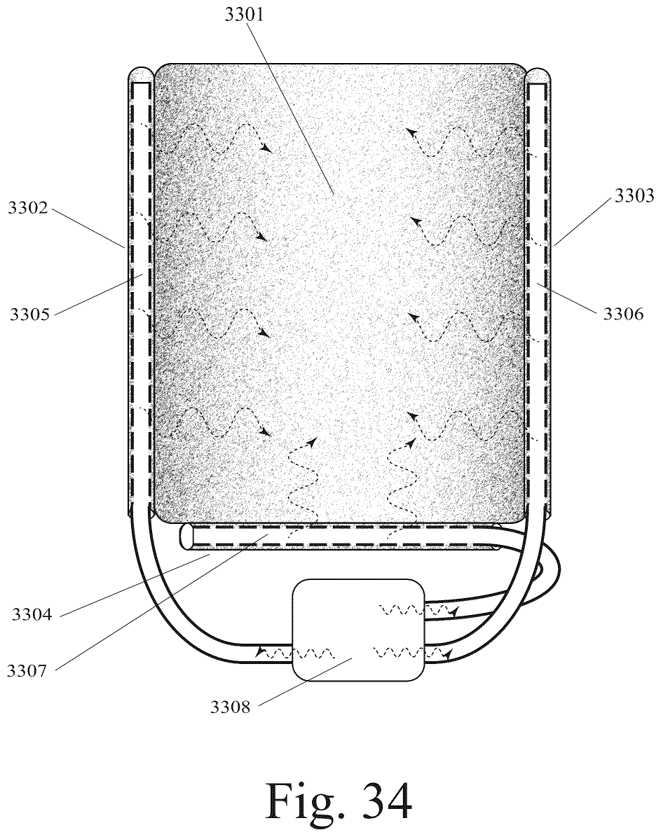

FIGS. 33 and 34 show two top-down views, at two different times, of a portion of a device or system which changes, manages, and/or controls the climate (e.g. temperature and/or humidity) of one or more areas (e.g. sides or zones) of a bed which comprises: a bedding component (e.g. a bottom sheet, top sheet, mattress, or mattress pad) with a plurality of longitudinal compartments (e.g. compartments, pockets, or channels) around (portions of) its perimeter, wherein one or more intra-bed airflow channels are removably inserted into the longitudinal compartments to deliver air to a space (e.g. between bottom sheet and top sheet) in the bed. FIG. 33 shows this portion of a device or system at first time, before the intra-bed flow channels have been inserted into the longitudinal channels on the perimeter of the bedding component. FIG. 34 shows this portion of a device or system at second time, after the intra-bed flow channels have been inserted into the longitudinal channels on the perimeter of the bedding component.

In an example, the intra-bed flow channels can be temporarily removed from the bedding component before the bedding component is washed. In an example, the longitudinal components can be made from air-permeable material such as soft, breathable fabric. In an example, a bedding component can be a fitted sheet which has longitudinal fabric channels around portions of its perimeter (e.g. on the lateral sides and foot of the bed) into which airflow channels (e.g. airflow tubes with holes) are removably inserted. In this example, there are three longitudinal components: one along the right side of the bed; one along the left side of the bed; and one along the foot of the bed. In this example, there are three intra-bed flow channels, one for insertion into each of the three longitudinal components.

In an example, a device or system to change the temperature and/or moisture level of a bed can comprise: a fitted sheet or mattress pad which is configured to lie over a bed mattress and under a person while they sleep; one or more longitudinal channels which are made from fabric and span at least a portion of the perimeter of the sheet or mattress pad; one or more longitudinal air conduits which are removably-inserted into the longitudinal channels, wherein there are openings and/or holes in the air conduits through which air flows out of the air conduits into the bed to change the temperature and/or moisture level of the bed; and a flow accelerator which accelerates a flow of air into the longitudinal air conduits.

In an example, the one or more longitudinal channels on the perimeter of the sheet or mattress pad can further comprise: a first channel along the upper right side of the sheet or mattress pad; a second channel along the upper left side of the sheet or mattress pad; and a third channel along the foot of the sheet or mattress pad. In an example, the one or more longitudinal air conduits on the perimeter of the sheet or mattress pad can further comprise: a first air conduit along the upper right side of the sheet or mattress pad; a second air conduit along the upper left side of the sheet or mattress pad; and a third air conduit along the foot of the sheet or mattress pad.

In an example, the one or more longitudinal air conduits on the perimeter of the sheet or mattress pad can further comprise: a first air conduit which is inserted into a first fabric channel along the upper right side of the sheet or mattress pad; a second air conduit which is inserted into a second fabric channel along the upper left side of the sheet or mattress pad; and a third air conduit which is inserted into a third fabric channel along the foot of the sheet or mattress pad.

With respect to specific components, FIGS. 33 and 34 show a portion of a device or system which changes, manages, and/or controls the climate (e.g. temperature and/or humidity) of one or more areas (e.g. sides or zones) of a bed which comprising: a bedding component 3301 (e.g. a bottom sheet, top sheet, mattress, or mattress pad) with a plurality of longitudinal compartments 3302, 3303, and 3304 (e.g. compartments, pockets, or channels) around (portions of) its perimeter; one or more intra-bed airflow channels 3305, 3306, and 3307 are removably inserted into the longitudinal compartments to deliver air to a space (e.g. between bottom sheet and top sheet) in the bed; and a housing 3308 which contains a flow-accelerating unit and/or a heat exchange unit. Example variations discussed elsewhere in this disclosure or priority-linked disclosures can also be applied to this example where relevant.

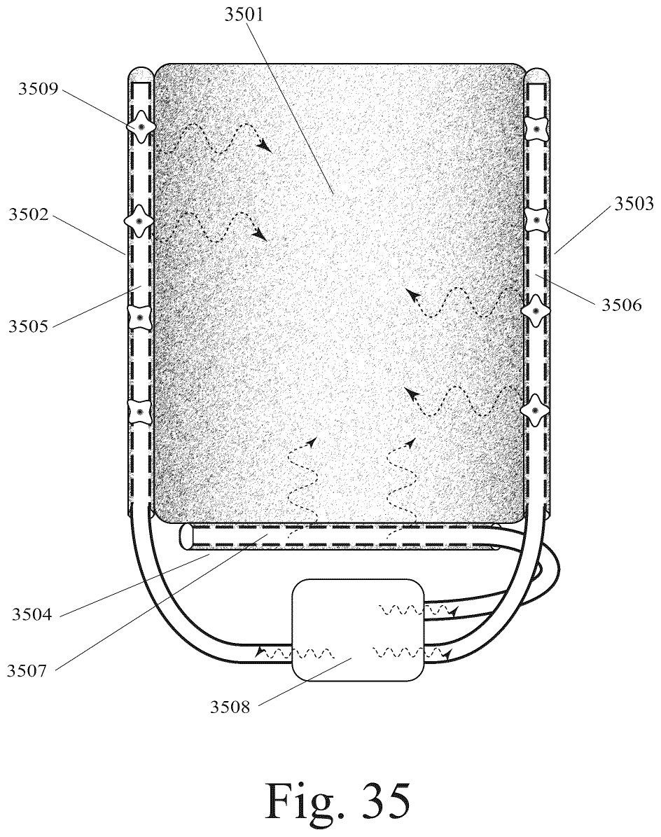

Fig. shows a two top-down view of a portion of a device or system which changes, manages, and/or controls the climate (e.g. temperature and/or humidity) of one or more areas (e.g. sides or zones) of a bed like the one in FIG. 34, except that it also includes a plurality of airflow valves which enable customized adjustment of airflow to different areas (e.g. sides or zones) of the bed. In FIG. 35, these valves have been adjusted to direct airflow to the upper body of a (first) person on left side of the bed and to direct airflow to the lower body of a (second) person on the right side of the bed. In other examples, a wide variety of relative airflow patterns can be created by adjusting individual valves in accordance with personal preferences.

With respect to specific components, FIG. 35 shows a portion of a device or system which changes, manages, and/or controls the climate (e.g. temperature and/or humidity) of one or more areas (e.g. sides or zones) of a bed which comprising: a bedding component 3501 (e.g. a bottom sheet, top sheet, mattress, or mattress pad) with a plurality of longitudinal compartments 3502, 3503, and 3504 (e.g. compartments, pockets, or channels) around (portions of) its perimeter; one or more intra-bed airflow channels 3505, 3506, and 3507 are removably inserted into the longitudinal compartments to deliver air to a space (e.g. between bottom sheet and top sheet) in the bed; a housing 3508 which contains a flow-accelerating unit and/or a heat exchange unit; and a plurality of airflow valves (including 3509). Example variations discussed elsewhere in this disclosure or priority-linked disclosures can also be applied to this example where relevant.

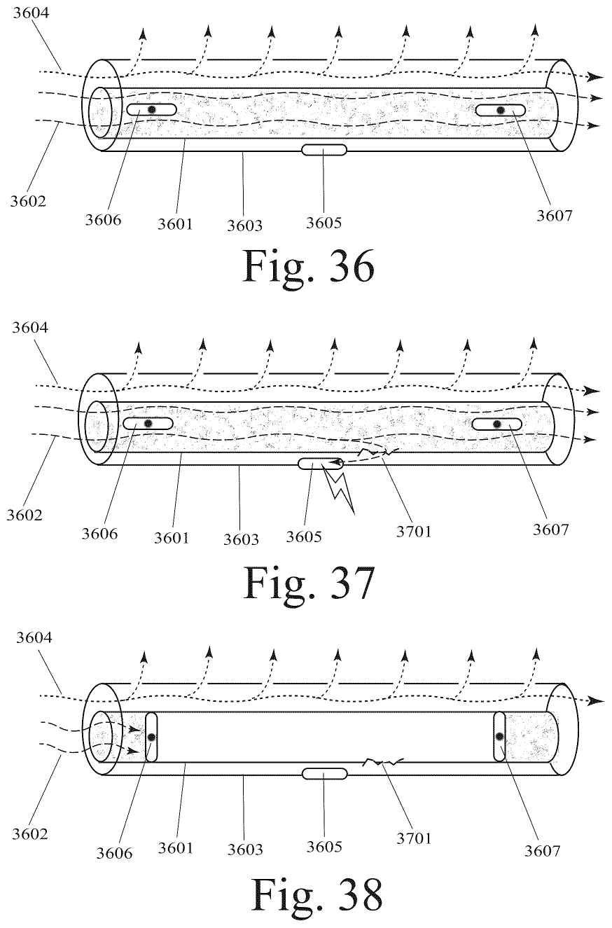

FIGS. 36 through 38 show three cross-sectional views, at three different times, of a leakage mitigation design for a hybrid device or system which uses both liquid flow and air flow through a bedding component to change, manage, and/or control the climate (e.g. temperature and/or humidity) of one or more areas (e.g. sides or zones) of a bed.

In this example, a liquid flow channel is inside (e.g. nested within) an air flow channel through a bedding component. In this example, the liquid flow cools (or heats) the air flow and then the cooled (or heated) air is delivered through openings (e.g. holes) in the airflow channel into a bed space (e.g. the space between a bottom sheet and a top sheet). This design further comprises: a plurality of valves which can close off specific sections of the liquid flow channel; and a plurality of moisture sensors in sections of the airflow channel. If a moisture sensor in a particular section of the airflow channel detects a leak 3701 from the section of the liquid flow channel inside it, then this triggers valves to close off that particular section of the liquid flow channel in order to mitigate leakage.

With respect to specific components, the leakage mitigation design shown in FIGS. 36 through 38 comprises: an intra-bed liquid flow channel 3601; a liquid 3602 (e.g. water) which flows through the intra-bed liquid flow channel; an intra-bed air flow channel 3603, wherein the intra-bed liquid flow channel is in thermal communication with (e.g. nested within) the intra-bed air flow channel; an air flow 3604 which flows through the intra-bed air flow channel, wherein there are openings (e.g. holes) in a portion (e.g. in the upper perimeter) of the intra-bed air flow channel through which the air flow escapes into a bed space (e.g. the space between a bottom sheet and a top sheet); one or more moisture (e.g. moisture or humidity) sensors 3605 in fluid communication with the intra-bed air flow channel; and a plurality of valves 3606 and 3607 in fluid communication with the intra-bed liquid flow channel, wherein detection of moisture by analysis of data from the one or more moisture sensors triggers one or more valves to close off (e.g. isolate) one or more sections of the intra-bed liquid flow channel in order to mitigate leakage of liquid from the intra-bed flow channel. Example variations discussed elsewhere in this disclosure or priority-linked disclosures can also be applied to this example where relevant.

In an example, a device or system to control the climate of one or more areas of a bed can comprise: one or more intra-room flow channels which conduct a flowable substance between a first component and a second component in a bedroom; wherein the first component is selected from the group consisting of (a) an airflow vent though which air enters the bedroom from a central HVAC (Heating Ventilation and Air Conditioning) system, (b) an airflow vent through which air exits the bedroom to a central HVAC system, and (c) an air conditioner mounted in a bedroom window; and wherein the second component is a bedding component; and one or more intra-bed flow channels which conduct a flowable substance through the bedding component in order to change the climate of one or more areas of a bed.

In an example, the flowable substance conducted by one or more intra-room flow channels can be air and the flowable substance conducted by one or more intra-bed flow channels can also be air. In an example, the flowable substance conducted by one or more intra-room flow channels can be a liquid and the flowable substance conducted by one or more intra-bed flow channels can also be the liquid. In an example, the flowable substance conducted by one or more intra-room flow channels can be air and the flowable substance conducted by one or more intra-bed flow channels can be a liquid. In an example, the flowable substance conducted by one or more intra-room flow channels can be a liquid and the flowable substance conducted by one or more intra-bed flow channels can be air.

In an example, the device or system can further comprise a valve which changes the proportion of air from a central HVAC vent which flows into the intra-room flow channel versus the proportion of air from the central HVAC which flows into ambient air in the bedroom. In an example, the one or more intra-room flow channels can comprise one or more modular sections. In an example, the one or more intra-room flow channels can comprise one or more modular sections selected from the group consisting of: a first type of intra-room flow channel modular section which spans straight along a wall; a second type of intra-room flow channel modular section which spans around a corner where two walls intersect; and a third type of intra-room flow channel modular section which at least partially covers a central HVAC vent.

In an example, the bedding component can be selected from the group consisting of: bed mattress, bed mattress pad or topper, lower/fitted bed sheet, upper bed sheet, blanket, comforter, quilt, and other specialized bedding. In an example, the one or more intra-bed flow channels can span at least a portion of the perimeter of the bed. In an example, the one or more intra-bed flow channels can span a right side, a left side, and a foot side of the perimeter of the bed. In an example, the bedding component can have longitudinal fabric channels along at least a portion of the perimeter of the bed into which one or more intra-bed flow channels are inserted. In an example, the bedding component can be a lower/fitted bed sheet with longitudinal fabric channels around its right side, its left side, and its foot side and wherein one or more intra-bed flow channels are inserted into the longitudinal fabric channels.

In an example, the device or system can further comprise a plurality of valves which are in fluid communication with the one or more intra-bed flow channels, wherein the valves are manually opened or closed by turning a knob, pivoting a lever, rotating a threaded member, sliding a strip, or placing a cap. In an example, the device or system can further comprise a plurality of valves in fluid communication with the one or more intra-bed flow channels, wherein the valves are automatically opened or closed by an electric actuator, hydraulic actuator, or pneumatic actuator.

In an example, the device or system can further comprise one or more sensors selected from the group consisting of: temperature sensor, pressure sensor, force sensor, moisture sensor, humidity sensor, motion sensor, sound sensor, light sensor, optical sensor, infrared sensor, spectroscopic sensor, electromagnetic energy sensor, EEG sensor, and EMG sensor.

In an example, a device or system to control the climate of one or more areas of a bed can comprise: a first intra-bed flow channel, wherein the first intra-bed flow channel conducts a liquid through a bedding component; and a second intra-bed flow channel, wherein the second intra-bed flow channel conducts air through the bedding component, and wherein the first intra-bed flow channel is inside the second intra-bed flow channel. In an example, there can be holes and/or openings in the second intra-bed flow channel through which air escapes.

In an example, a leak reduction mechanism for a device or system which controls the climate of one or more areas of a bed can comprise: an intra-bed flow channel which conducts a liquid through a bedding component; one or more valves which are in fluid communication with the intra-bed flow channel; one or more leak detection sensors selected from the group consisting of (a) a moisture and/or humidity sensor under the section of the intra-bed flow channel, (b) a pressure sensor which is in fluid communication with the section of the intra-bed flow channel, and (c) a fluid volume sensor which is in fluid communication with the section of the intra-bed flow channel; wherein the one or more valves are automatically closed if data from the one or more sensors indicates liquid leaking from the section. In an example, there can be a first valve at a first end of the section and a second valve at a second end of the section, wherein both valves are closed if data from the one or more sensors indicates liquid leaking from the section.

In an example, a device or system to change the temperature and/or moisture level of a bed can comprise: a fitted sheet or mattress pad which is configured to lie over a bed mattress and under a person while they sleep; one or more longitudinal channels which are made from fabric and span at least a portion of the perimeter of the sheet or mattress pad; one or more longitudinal air conduits which are removably-inserted into the longitudinal channels, wherein there are openings and/or holes in the air conduits through which air flows out of the air conduits into the bed to change the temperature and/or moisture level of the bed; and a flow accelerator which accelerates a flow of air into the longitudinal air conduits.

In an example, the one or more longitudinal channels on the perimeter of the sheet or mattress pad can further comprise: a first channel along the upper right side of the sheet or mattress pad; a second channel along the upper left side of the sheet or mattress pad; and a third channel along the foot of the sheet or mattress pad. In an example, the one or more longitudinal air conduits on the perimeter of the sheet or mattress pad can further comprise: a first air conduit along the upper right side of the sheet or mattress pad; a second air conduit along the upper left side of the sheet or mattress pad; and a third air conduit along the foot of the sheet or mattress pad.

In an example, the one or more longitudinal air conduits on the perimeter of the sheet or mattress pad can further comprise: a first air conduit which is inserted into a first fabric channel along the upper right side of the sheet or mattress pad; a second air conduit which is inserted into a second fabric channel along the upper left side of the sheet or mattress pad; and a third air conduit which is inserted into a third fabric channel along the foot of the sheet or mattress pad.

I claim:

1. A device or system to control the climate of one or more areas of a bed comprising:
one or more intra-room flow channels which conduct a flowable substance between a first component and a second component in a bedroom; wherein the first component is selected from the group consisting of (a) an airflow vent though which air enters the bedroom from a central HVAC system, (b) an airflow vent through which air exits the bedroom to a central HVAC system, and (c) an air conditioner mounted in a bedroom window; and wherein the second component is a bedding component; and
one or more intra-bed flow channels which conduct a flowable substance through the bedding component in order to change the climate of one or more areas of a bed.

2. The device or system in claim 1 wherein the flowable substance conducted by one or more intra-room flow channels is air and the flowable substance conducted by one or more intra-bed flow channels is also air.

3. The device or system in claim 1 wherein the flowable substance conducted by one or more intra-room flow channels is a liquid and the flowable substance conducted by one or more intra-bed flow channels is also the liquid.

4. The device or system in claim 1 wherein the flowable substance conducted by one or more intra-room flow channels is air and the flowable substance conducted by one or more intra-bed flow channels is a liquid.

5. The device or system in claim 1 wherein the flowable substance conducted by one or more intra-room flow channels is a liquid and the flowable substance conducted by one or more intra-bed flow channels is air.

6. The device or system in claim 1 wherein the device or system further comprises a valve which changes the proportion of air from a central HVAC vent which flows into the intra-room flow channel versus the proportion of air from the central HVAC which flows into ambient air in the bedroom.

7. The device or system in claim 1 wherein the one or more intra-room flow channels comprise one or more modular sections.

8. The device or system in claim 7 wherein the one or more intra-room flow channels comprise one or more modular sections selected from the group consisting of: a first type of intra-room flow channel modular section which spans straight along a wall; a second type of intra-room flow channel modular section which spans around a corner where two walls intersect; and a third type of intra-room flow channel modular section which at least partially covers a central HVAC vent.

9. The device or system in claim 1 wherein the bedding component is selected from the group consisting of: bed mattress, bed mattress pad or topper, lower/fitted bed sheet, upper bed sheet, blanket, comforter, quilt, and other specialized bedding.

10. The device or system in claim 1 wherein one or more intra-bed flow channels span at least a portion of the perimeter of the bed.

11. The device or system in claim 10 wherein one or more intra-bed flow channels span a right side, a left side, and a foot side of the perimeter of the bed.

12. The device or system in claim 10 wherein a bedding component has longitudinal fabric channels along at least a portion of the perimeter of the bed into which one or more intra-bed flow channels are inserted.

13. The device or system in claim 12 wherein the bedding component is a lower/fitted bed sheet with longitudinal fabric channels around its right side, its left side, and its foot side and wherein one or more intra-bed flow channels are inserted into the longitudinal fabric channels.

14. The device or system in claim 1 wherein the device or system further comprises a plurality of valves which are in fluid communication with the one or more intra-bed flow channels, wherein the valves are manually opened or closed by turning a knob, pivoting a lever, rotating a threaded member, sliding a strip, or placing a cap.

15. The device or system in claim 1 wherein the device or system further comprises a plurality of valves in fluid communication with the one or more intra-bed flow channels, wherein the valves are automatically opened or closed by an electric actuator, hydraulic actuator, or pneumatic actuator.

16. The device or system in claim 1 wherein the device or system further comprises one or more sensors selected from the group consisting of: temperature sensor, pressure sensor, force sensor, moisture sensor, humidity sensor, motion sensor, sound sensor, light sensor, optical sensor, infrared sensor, spectroscopic sensor, electromagnetic energy sensor, EEG sensor, and EMG sensor.

17. A device or system to control the climate of one or more areas of a bed comprising:
a first intra-bed flow channel, wherein the first intra-bed flow channel conducts a liquid through a bedding component; and
a second intra-bed flow channel, wherein the second intra-bed flow channel conducts air through the bedding component, and wherein the first intra-bed flow channel is inside the second intra-bed flow channel.

18. The device or system in claim 17 wherein there are holes and/or openings in the second intra-bed flow channel through which air escapes.

19. A leak reduction mechanism for a device or system which controls the climate of one or more areas of a bed comprising:
an intra-bed flow channel which conducts a liquid through a bedding component;
one or more valves which are in fluid communication with the intra-bed flow channel;
one or more leak detection sensors selected from the group consisting of (a) a moisture and/or humidity sensor under the section of the intra-bed flow channel, (b) a pressure sensor which is in fluid communication with the section of the intra-bed flow channel, and (c) a fluid volume sensor which is in fluid communication with the section of the intra-bed flow channel;
wherein the one or more valves are automatically closed if data from the one or more sensors indicates liquid leaking from the section.

20. The leak reduction mechanism in claim 19 where a first valve is at a first end of the section, a second valve is at a second end of the section, and both valves are closed if data from the one or more sensors indicates liquid leaking from the section.
